(19) United States
(12) Patent Application Publication
(10) Pub. No.: US 2005/0063989 A1
(43) Pub. Date: Mar. 24, 2005

(54) Ion channel hybrid proteins as electrical sensors and use thereof

(75) Inventors: Renaud Derand, Luxembourg (LU); Elisabeth Garcia, Echirolles (FR); Anne-Lise Prost, Lons-Le-Saunier (FR); Jean Revilloud, Saint-Martin-Le-Vinoux (FR); Michel Vivaudou, Saint-Egreve (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: COMMISSARIAT A L'ENERGIE ATOMIQUE, Paris (FR)

(21) Appl. No.: 10/665,283

(22) Filed: Sep. 22, 2003

(51) Int. Cl. C12Q 1/68; C07H 21/04; A61K 39/00; C07K 14/705

(52) U.S. Cl. 424/192.1; 530/350; 435/69.1; 435/320.1; 435/325; 536/23.5

(57) ABSTRACT

Hybrid proteins associating a membrane protein with an ion channel which is not naturally coupled to said membrane protein and their use as electrical sensors of said membrane protein (receptor, transporter) activity, for studying said membrane proteins, and for detecting and screening molecules of interest.

The application relates to hybrid proteins associating a membrane protein with an ion channel and to their use as electrical sensors of said membrane protein (receptor, transporter) activity, for studying said membrane proteins, and for detecting and screening molecules of interest.

Due to their ability to bind ligands selectively with high affinity, membrane proteins (receptors, transporters) represent, in theory, biochemical probes (bioprobes) or biochemical sensors (biosensors) useful for the detection and the screening of various molecules; use of biosensors cover numerous areas including environmental safety and food quality (detection of pollutants/contaminants), as well as human health [medical diagnostics (detection of microorganisms, toxics.) and therapeutics (drug discovery)].

However, in practice, the use of biosensors to detect molecules of interest is limited due to the lack of the sensitivity and the difficulties to prepare said biosensors.

More precisely:
the detection of ligand binding to the biosensor requires high amounts of purified proteins which are particularly difficult to obtain with membrane proteins, and
the detection of the molecules of interest requires highly sensitive biosensors since most of the events involving hazards in human health (i.e. airborne droplets/dust transmission routes), in environmental safety and in food quality occur when causative agents are at very low concentration (down to atto-molar (10⁻¹⁸M) to zeptomolar (10⁻²¹M) or even below).

Therefore, there is a need for new biosensors which are more sensitive and easier to prepare.

Ion channels are cellular membrane proteins that mediate a wide variety of physiological functions including rapid signaling, excitability and transport. They represent highly sensitive electrical sensors since the current pulses of single channels which are in the order of a few picoamperes lasting a few milliseconds, are yet detectable by conventional electrophysiological techniques.

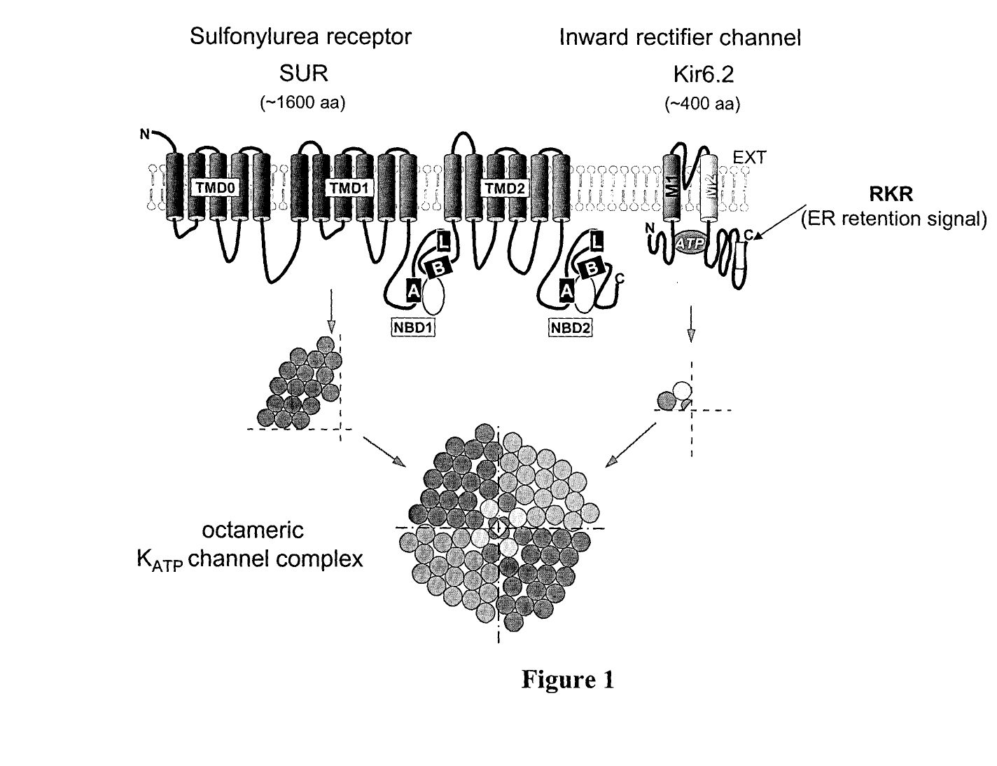

The sulfonylurea receptor (SUR) from the ABC (ATP-binding cassette) transporter family is unique in that it is functionally coupled with an ion channel (Kir6.2 protein from the inner rectifier K⁺ channel family) to form an ATP-sensitive potassium channel, best known in pancreatic β-cells for its key role in the cascade linking glycemic levels to insulin secretion (Seino, 1999, Annu. Rev. Physiol., 61, 337-362 ; Terzic A and Vivaudou M, 2001, In. Potassium channels in Cardiovascular Biology, Eds S. L. Archer and N.J. Rusch, Kluwer Academic/Plenum Publishers, New York, pp257-277). Four Kir6.2 subunits form a pore which can be inhibited by ATP whereas SUR proteins act as peripheral ADP-sensing regulators (FIG. 1). A specific interaction of Kir6.2 and SUR is required for surface expression of K_{ATP} channels since both subunits contain ER retention signals preventing targeting to the cell surface of all but the 4:4 stoichiometry where all signals are masked.

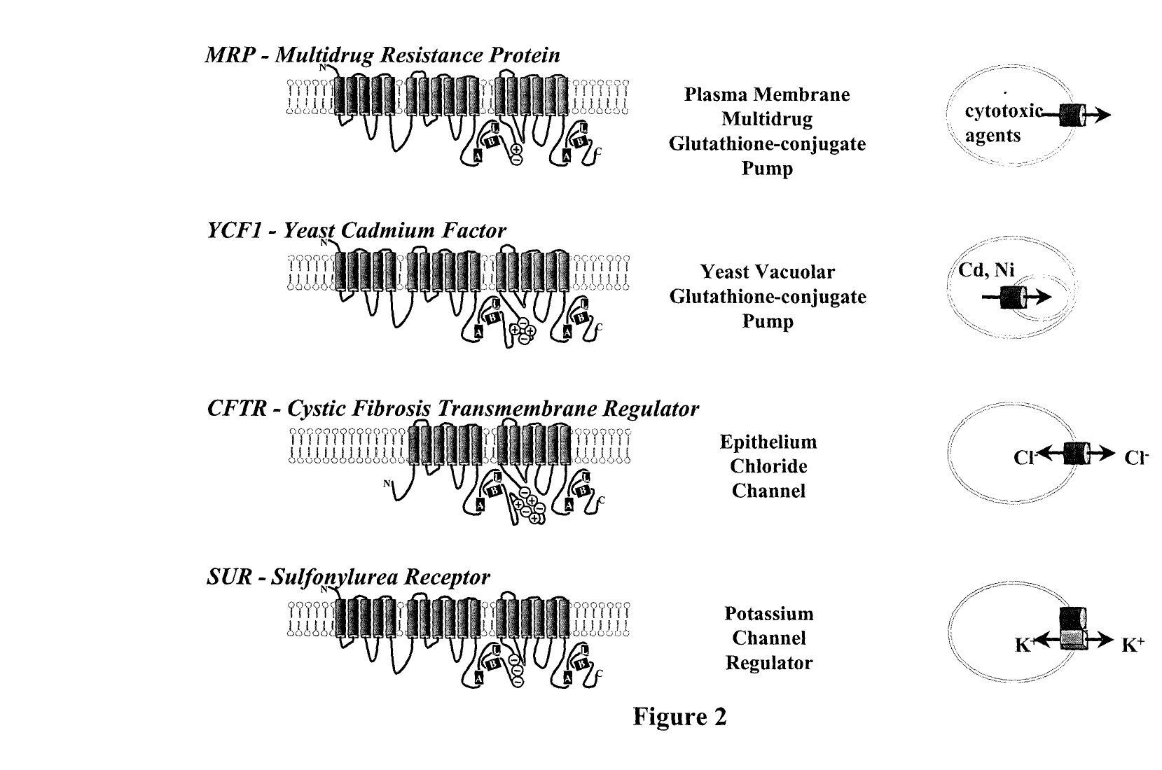

In spite of sequence homology with SUR, other members of the ABC family such as Mdr1 (Multidrug resistance 1) or the MRP or ABCC subfamily of ABC transporters such as CFTR (Cystic Fibrosis Transmembrane Regulator; epithelium chloride channel), MRP (Plasma Membrane multidrug, Glutathione-conjugate pump; cytotoxic agents transporters) or YCF1 (Yeast cadmium factor; heavy metal transporter: cadmium, nickel, mercury, arsenite) are active transporters but are not physiologically coupled to ion channels and, except for CFTR, have therefore no ion channel function (FIG. 2).

Furthermore, most of the receptors and transporters which are potential targets for the screening of new drugs and/or the detection of molecules of interest are not physiologically coupled to ion channels.

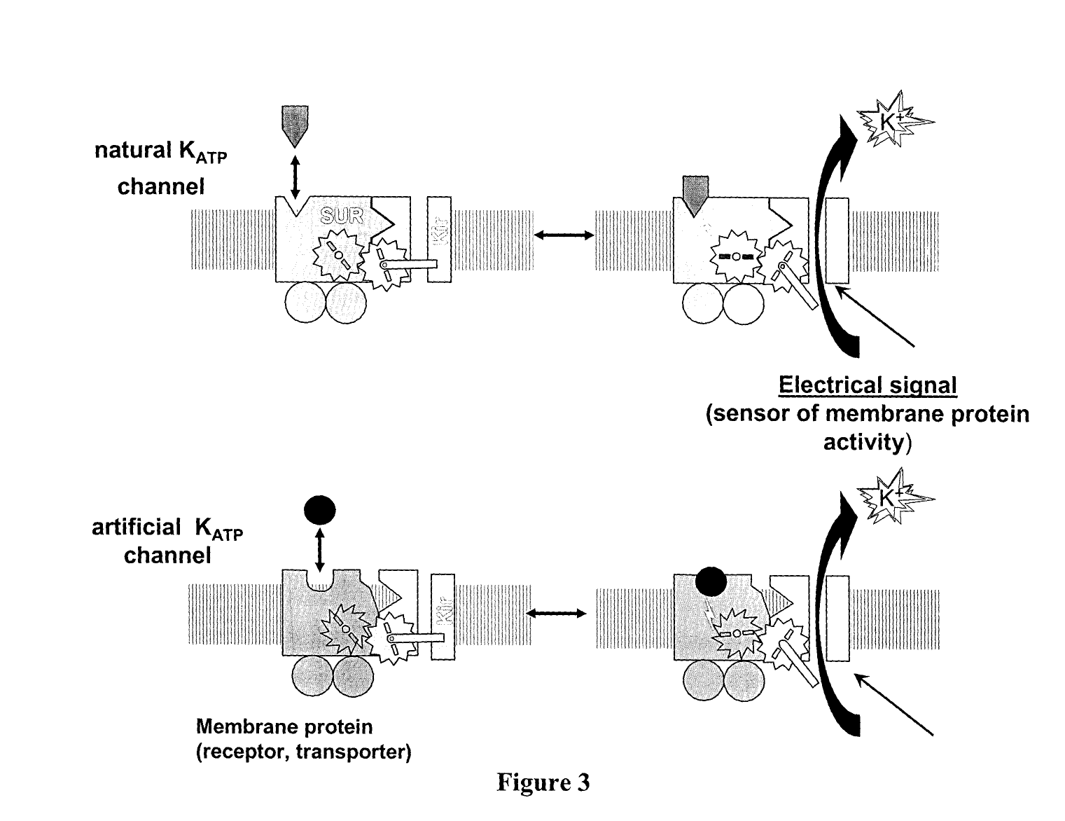

The inventors have constructed fusions of Kir6.2 with various membrane proteins (receptors/transporters), expressed the fusions in Xenopus oocytes, and assessed the functional coupling between the membrane protein and the receptor by testing the effect of specific ligands/substrates of said membrane proteins on channel gating. Using SUR-Kir6.2 fusion as a control, they have demonstrated that fusions of Kir6.2 with ABC transporters and more distantly related membrane proteins form a functional channel; in the ion channel-receptor/transporter hybrid protein, the receptor/transporter occupancy by the ligand/substrate is transferred to the ion channel (Kir6.2) and transduced into an electrical signal that is detected by standard electrophysiological techniques (patch clamp technique, two-microelectrode voltage clamp). These results demonstrate that ion channel hybrid proteins represent electrical sensors of the activity of membrane proteins (FIG. 3).

These electrical sensors are highly sensitive and easy to prepare since the signal of a single channel molecule is detectable and registered by standard electrophysiological techniques.

These improvements allow the use of ion channel hybrid proteins as electrical sensors to detect molecules which are present at very low concentrations (pollutants/ contaminants) or to screen a large number of molecules of therapeutic interest, simultaneously (drug discovery). In addition, these electrical sensors represent very convenient tools for basic research since they allow structure-function studies of any membrane protein from a single molecule.

Thus, the invention relates to an hybrid protein consisting essentially of the fusion of a membrane protein with an ion channel which is not naturally coupled to said membrane protein.

By “not naturally coupled”, it is intended that unlike SUTR and Kir6.2, said membrane protein and said ion channel are not naturally associated in cells; said membrane protein and said ion channel, when co-expressed in a cell, do not form complexes which can be detected at the cell membrane, by any appropriate mean, known in the art.

The invention includes hybrid proteins deriving from membrane proteins such as receptors and transporters (active and passive transporters or pumps). Examples of such receptors include with no limitation: neurotransmitter receptors, hormone receptors, drug receptors and olfactive receptors. Example of such transporters are heavy metal transporters.

Accordingly, the invention includes hybrid proteins deriving from ion channels such as, non exhaustively: K+ channels like members of the Kir and the Kv families, mechanosensitive channels like MscL, Na⁺ channels and Ca2⁺ channels. Preferred channels include those which have one or several of the following properties: they are coupled with a receptor/transporter in a physiological manner, they are encoded by a very small gene and easily handled by molecular biology, their gating behavior is straightforward and they are regulated and blocked by a simple ligand; in an heterologous expression system, this ligand allows testing of the functionality of the hybrid protein by simple electrophysiological assays. For example, Kir6.2 is easily tested by addition of ATP. If a voltage-dependent channel is used, pulses can be used for that purpose.

The invention includes hybrid proteins comprising any functional derivative of the membrane protein and the ion channel as defined above. By “functional derivative” it is intended a membrane protein or an ion channel comprising one or more modifications which do not affect substantially the activity of said membrane protein (receptor/transporter) or said ion channel.

Such modifications include, non exhaustively, those which have the following effect on the resulting hybrid protein : increase of the level of expression, increase of the cell surface expression, improvement of the purification, improvement of the detection, increase of the association and functional coupling between the ion channel and the membrane protein, increase of the stability in membranes (natural/artificial), increase/decrease of the affinity of the ligand/substrate to the receptor/transporter, increase/decrease of the ion selectivity and unitary current of the ion channel.

They include: addition and/or deletion and/or substitution of one or more amino acid residue in the peptide chain, and/or replacement of one or more of the amide bond by a non-amide bond, and/or replacement of one or more amino acid side chain by a different chemical moiety, and/or protection of the N-terminus, the C-terminus, or one or more of the side chain by a protecting group, and/or introduction of double bonds and/or cyclization and/or stereospecificity into the amino acid chain.

For example, it is possible to substitute amino acids by equivalent amino acids. “Equivalent amino acid” is used herein to name any amino acid that may substitute for one of the amino acids belonging to the initial protein structure without modifying the biological activity of the initial protein structure. These equivalent amino acids may be determined by their structural homology with the initial amino acids to be replaced and by their biological activity on the ligand/substrate of the membrane protein or ion channel according to the invention. As an illustrative example, it should be mentioned the possibility of carrying out substitutions like, for example, leucine by valine or isoleucine, aspartic acid by glutamic acid, glutamine by asparagine, asparagine by lysine etc., it being understood that the reverse substitutions are permitted in the same conditions. In some cases, it may also be possible to replace of a residue in the L-form by a residue in the D-form or the replacement of the glutamine (Q) residue by a pyro-glutamic acid compound.

To increase the cell surface expression of the hybrid protein, it is possible to mutate or delete sequences known to decrease trafficking to the cell plasma membrane such as endoplasmic reticulum (ER) retention signal(s) and/or to insert sequences known to increase trafficking to the cell plasma membrane such as export signals; RKR retention signals may be mutated to RAA, and/or FCYENE export signal may be inserted in the hybrid protein.

To improve the purification of the hybrid protein, it is possible to introduce well-known tags such as epitopes (hemagglutinine epitope, Myc epitope, FLAG epitope) or a polyhistidine sequence.

To increase the association and the coupling between the ion channel and the receptor protein, it is possible to incorporate, into the membrane protein, regions of natural partners which interact with the ion channel. Example of such regions include the TMD0 domain of SUR1 or SUR2A.

To increase the affinity of the ligand/substrate to the membrane protein (receptor/transporter), it possible to mutate amino acids from the binding site of said ligand/substrate.

Since all the modifications are known in the art, it is submitted that a person skilled in the art will be able to produce, test, identify and select the proteins according to the present invention.

The hybrid protein according to the invention has the properties of an electrical sensor:
it forms an artificial ion channel wherein the membrane protein (receptor/transporter) is functionally coupled with the ion channel so that ligand/substrate binding to the membrane protein (receptor/transporter) induces conformational changes in the membrane protein which are allosterically transmitted to the channel, yielding changes in channel gating (ion current) measurable by standard electrophysiological techniques.

The hybrid protein is either expressed at the surface of an host cell, or it is incorporated in an artificial membrane.

Surface expression of the hybrid protein is assessed by techniques well known in the art. For example, the hybrid protein comprises an epitope which allows cell surface detection of said hybrid protein with appropriate antibodies, for example by chemoluminescence techniques.

Ion channel function is assessed using the patch-clamp technique in inside-out and outside-out configurations as well as with the two-electrode voltage clamp on the whole cell. Functional coupling between membrane protein and channel is assessed by testing the effect of ligands/substrates of the membrane protein (transporter/receptor) on channel gating.

The relative positions of the membrane protein and the ion channel in the hybrid protein are determined by the orientation of said proteins in the plasma membrane ; the extremities which are fused must be on the same side of the plasma membrane (cytolasmic or extracellular). For example the N-terminus and the C-terminus of Kir6.2 which are cytolasmic can be fused to the extremity of a membrane protein which is also cytoplasmic. In the case of MRP1, the N-terminus is extracellular and the C-terminus cytoplasmic and thus the C-terminus of MRP1 is fused to the N-terminus of Kir6.2.

According to an advantageous embodiment of the invention, said hybrid protein comprises a spacer between the membrane protein and the ion channel.

According to the invention, “spacer” means a peptide which favors the flexibility and/or functional coupling between the ion channel and the membrane protein. A spacer containing relatively neutral amino acid residues such as glycine, serine, alanine or glutamine may be used to increase the flexibility of the hybrid protein. Examples of such spacer include, non exhaustively, a peptide consisting of six glycine (hexaglycine) or ten glutamine residues.

According to another advantageous embodiment of the invention, said hybrid protein comprises a tag, for example an epitope, to facilitate its detection and/or its purification.

According to another advantageous embodiment of the invention, said membrane protein is a receptor such as an hormone receptor, an olfactive receptor or a receptor for a pollutant/contaminant, for example, the M2 muscarinic receptor, the β2 adrenergic receptor, or the OR-5, OR-17 or OR-23 olfactive receptor.

According to another advantageous embodiment of the invention, said membrane protein is a transporter such as an ABC transporter or a transporter for a pollutant/contaminant, for example Mdr1 or a transporter from the MRP class such as CFTR (Cystic fibrosis transmembrane regulator, MRP1 (Multidrug Resistance Protein), YCF1 (Yeast Cadmium Factor) and SUR (Sulfonylurea Transporter).

The expression “contaminant/pollutant” includes chemicals and pathogens (viruses, bacteria, spores, toxins, pollens) which are present in the environment and can be detected in the air, the soil, in food or in biological samples from humans or animals.

Preferably, said membrane protein is a heavy metal transporter, for example, YCF1 or CadA.

According to another advantageous embodiment of the invention, said ion channel is a potassium channel such as an ATP-sensitive K+ channel (K_{ATP}), for example from the Kir family. Examples of such proteins are Kir 6.2 fusions such as the fusions between Kir6.2 and an MRP protein presenting the sequence SEQ ID NO: 1 to 11.

Kir6.2 has the following advantages:
it is coupled with an ABC transporter in a physiological manner,
it is encoded by a very small gene (1200 bases) and easily handled by molecular biology,
its gating behaviour is straightforward : no voltage dependence, no tectification,
it is regulated and blocked by a simple ligand, ATP, that interacts with a cytoplasmic site without being hydrolysed. In an heterologous expression system, this ligand allows identification of the hydrid ion channel and in a reconstituted and non-oriented system, it could help to select the orientation of the channels to be studied.

Alternatively, other ion channels are used: simple voltage-dependent channel, for example, Kv1.x or mechanosensitive channels like MscL.

The hybrid protein of the present invention may be prepared by any suitable process. Preferably, it is obtained by genetic engineering technology. A typical example comprises: (1) amplifying a polynucleotide encoding the hybrid protein from known cDNAs representing the membrane protein and the ion channel, with appropriate primers deriving from said membrane protein and said ion channel sequences, (2) cloning said polynucleotide in an expression vector, (3) culturing a host cell containing said recombinant vector under conditions suitable for the expression of the hybrid protein at the surface of the host cell, and eventually, recovering the hybrid protein from the host cell culture.

The invention also provides a polynucleotide encoding the hybrid protein according to the invention, as well as the complement of said polynucleotide.

In particular, the invention provides the polynucleotide sequences encoding the hybrid proteins SEQ ID NO: 1 to 11, including all possible examples of nucleotide sequences encoding these peptides which result from the degeneration of the genetic code.

The invention also provides primers able to amplify said polynucleotides encoding the hybrid proteins SEQ ID NO: 1 to 11, which are selected from the group consisting of SEQ ID NO: 13 to 16 and 21, 22.

Nucleic acids of the invention may be obtained by the well-known methods of recombinant DNA technology and/or chemical DNA synthesis.

The invention also provides recombinant vectors comprising a polynucleotide encoding the hybrid protein of the invention. Vectors of the invention are preferably expression vectors, wherein a sequence encoding a hybrid protein of the invention is placed under control of appropriate transcriptional and translational control elements. These vectors may be obtained and introduced in a host cell by the well-known recombinant DNA and genetic engineering techniques.

The invention also provides a prokaryotic or eukaryotic host cell expressing a hybrid protein as defined above. Said host cell may be obtained by transfection with the hybrid protein, the polynucleotide (DNA, RNA) encoding said hybrid protein, or with the expression vector comprising said polynucleotide, as defined above.

The invention also relates to an electrical sensor comprising the hybrid protein as defined above, incorporated in a membrane.

According to the invention, said membrane may be a cell membrane or an artificial membrane.

The electrical sensor according to the invention is prepared from host cell expressing the hybrid protein as defined above, by using standard recombinant protein expression techniques, which are well-known in the art. Alternatively, the hybrid protein is purified and incorporated in an artificial membrane, by using standard techniques as described for example in Silvius J R, 1992, Annu. Rev. Biophys. Biomol. Struct., 21, 323-348.

The invention also relates to a method for assaying the activity of a membrane protein, comprising the step of:
bringing a ligand/substrate of said membrane protein in contact with an electrical sensor comprising an hybrid protein derived from said membrane protein, as defined above, and
measuring the resulting electrical signal by appropriate means.

The invention also relates to a method for the screening of an agonist of a membrane protein, comprising the step of:
bringing a drug to test in contact with an electrical sensor comprising an hybrid protein derived from said membrane protein, as defined above,
measuring the resulting electrical signal by appropriate means, and
selecting the drugs which induce an electrical signal.

The invention also relates to a method for the screening of an antagonist of a membrane protein, comprising the step of:
bringing a drug to test in contact with an electrical sensor comprising an hybrid protein derived from said membrane protein, as defined above, and with a ligand/substrate of said membrane protein,
measuring the resulting electrical signal by appropriate means, and
selecting the drugs which block the electrical signal induced by said ligand/substrate.

The electrical signal is measured using standard electrophysiological techniques such as the patch-clamp technique in inside-out and outside-out configurations as well as the two-electrode voltage clamp on a whole cell.

According to an advantageous embodiment of the screening method according to the invention, an electrical sensor comprising an hybrid protein derived from the MRP1 protein is used to screen anticancer drugs or multidrug reversing agents.

According to an advantageous embodiment of the screening method according to the invention, an electrical sensor comprising an hybrid protein derived from the SUR protein is used to screen antidiabetic, antiischemic or antihypertensive drugs.

The invention also relates to a method for the detection of a contaminant/pollutant, comprising the step of:
bringing a sample to be tested in contact with an electrical sensor comprising a membrane protein as defined above,
measuring the resulting electrical signal by appropriate means, and
detecting the presence of said contaminant/pollutant in said sample.

The electrical signal is measured using standard electrophysiological techniques as described above for the screening methods.

According to another advantageous embodiment of the detection method according to the invention, an electrical sensor comprising an hybrid protein derived from the YCF1 protein is used to detect heavy metals such as nickel, cadmium, arsenite and mercury.

The invention also relates to a kit for the screening of an agonist/antagonist of a receptor/transporter comprising at least one electrical sensor as defined above.

The invention also relates to a kit for the detection of a contaminant/pollutant comprising at least one electrical sensor as defined above.

The present invention will be further illustrated by the additional description and drawings which follow, which refer to examples illustrating the construction and the properties of the hybrid protein according to the invention. It should be understood however that these examples are given only by way of illustration of the invention and do not constitute in anyway a limitation thereof.

TABLE 1
List of sequences

| Sequence | SEQ ID NO: |
|---|---|
| MRP1-Kir6.2 | SEQ ID NO: 1 |
| YCF1-Kir6.2 | SEQ ID NO: 2 |
| SUR2A-Kir6.2 ΔC36 | SEQ ID NO: 3 |
| MRP1-Kir6.2ΔC36 | SEQ ID NO: 4 |
| YCF1-Kir6.2ΔC36 | SEQ ID NO: 5 |
| MRP1-Kir6.2[KR370AA] | SEQ ID NO: 6 |
| SUR2A-Kir6.2HA | SEQ ID NO: 7 |
| MRP1-Kir6.2HA | SEQ ID NO: 8 |
| YCF1-Kir6.2 ΔC36HA | SEQ ID NO: 9 |
| YCF1-Kir6.2 ΔC36HA- | SEQ ID NO: 10 |
| FCYENE | |
| Mdr1-Kir6.2 | SEQ ID NO: 11 |
| SUR2A-Kir6.2 | SEQ ID NO: 12 |
| MRP1 forward primer | SEQ ID NO: 13 |
| MRP1 fusion reverse primer | SEQ ID NO: 14 |
| Kir6.2 reverse primer | SEQ ID NO: 15 |
| Kir6.2 fusion forward primer | SEQ ID NO: 16 |
| SUR1 forward primer | SEQ ID NO: 17 |
| SUR1 fusion reverse primer | SEQ ID NO: 18 |
| SUR2A forward primer | SEQ ID NO: 19 |
| SUR2A fusion reverse primer | SEQ ID NO: 20 |
| YCF1 forward primer | SEQ ID NO: 21 |
| YCF1 fusion reverse primer | SEQ ID NO: 22 |
| HA epitope (YPYDVPDYA) | SEQ ID NO: 23 |
| DLYAYMEKGIT | SEQ ID NO: 24 |
| Export signal (FCYENE) | SEQ ID NO: 25 |

FIG. 1 illustrates the KATP channel structure. Kir6.2 and SUR are the constitutive subunits of the KATP channel. Four subunits of the inwardly rectifying K channel, Kir6.2, associate with four ATP binding cassette proteins, SUR, to form a functional K_{ATP} channel octamer. In Kir6.2, M1 and M2 represent the transmembrane helices. SUR posseses 3 transmembrane domains (TMD0, TMD1 and TMD2), and 2 cytoplasmic nucleotide binding domains (NBD1 and NBD2) incorporating the Walker A, Walker B and Linker L consensus sequences.

FIG. 2 illustrates the structure and function of several members of the ABCC or MRP subclass within the ABC transporter family.

FIG. 3 illustrates the principle of the electrical sensors according to the invention using K_{ATP} channels as an example. The hybrid protein wherein the membrane protein (receptor, transporter) is artificially coupled with Kir6.2 mimics the K_{ATP} channel wherein SUR is naturally coupled with Kir6. Thus, the conformational changes following ligand/substrate (drug, contaminant/pollutant . . . ) binding to the membrane protein are transferred to Kir6.2 and resulting ion currents are detected with electrophysiological techniques.

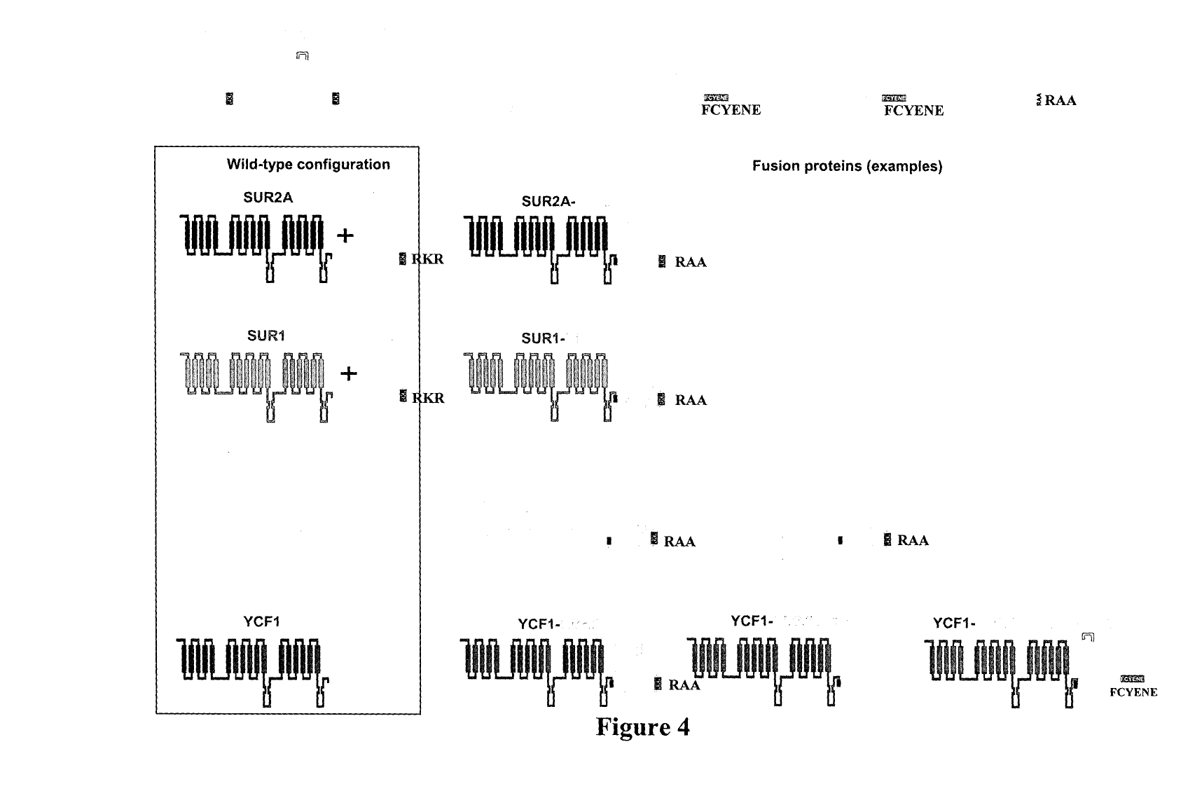

FIG. 4 illustrates the different ABC transporter/Kir6.2 hybrid protein constructs which have been studied.

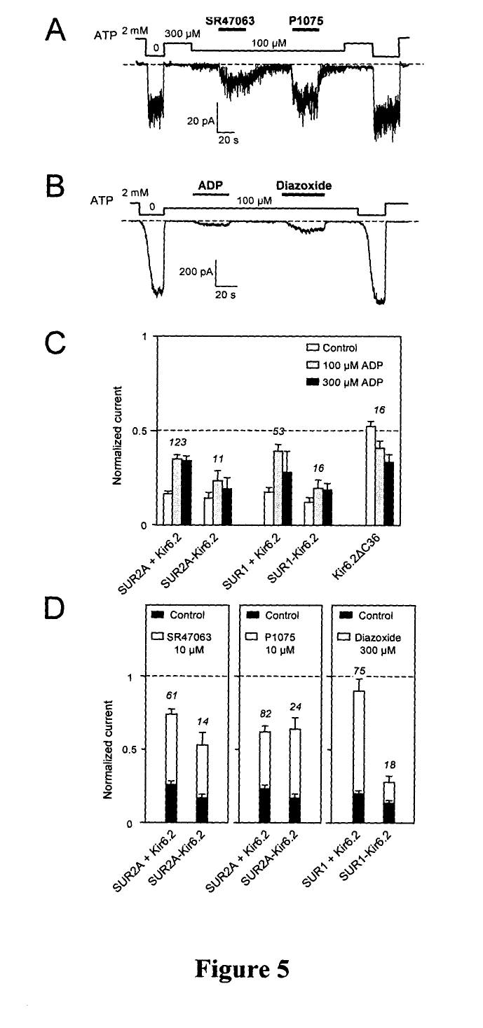

FIG. 5 illustrates the functional expression of SUR1-Kir6.2 and SUR2A-Kir6.2 fusion proteins. (A) Representative patch-clamp recording of SUR2A-Kir6.2 channels showing activation by SR47063 (10 μM) and P1075 (10 μM). (B) Idem with SUR1-Kir6.2 showing activation by ADP (300 μM) and diazoxide (300 μM). (C) Overall responses of tandem channels to ADP. Data from wild-type channels and from Kir6.2AC36 alone are included for comparison. (D) Idem with K_{ATP} channel openers. Data from wild-type channels are included for comparison. Numbers above the bars represent the number of patches considered.

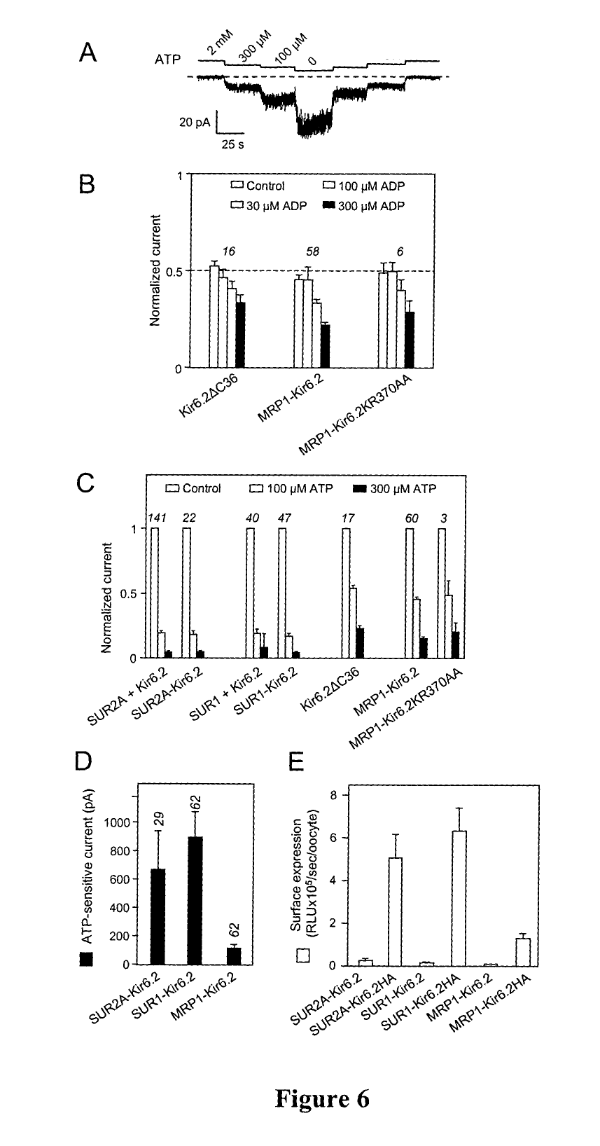

FIG. 6 illustrates the functional expression of MRP1-Kir6.2 fusion proteins. (A) Representative patch-clamp recording of MRP1-Kir6.2 channels. (B) At concentrations which produce activation of wild-type KATP channels, ADP applied in the presence of 100 μM ATP inhibits MRP1-Kir6.2 channels. Kir6.2AC36 and MRP1-Kir6.2KR370AA, which are devoid of the RKR retention signal, respond in a similar manner. (C) ATP inhibitory responses of the various wild-type, mutant and fusion channels demonstrate a depressed ATP sensitivity of MRP1-Kir6.2 channels comparable to that of Kir6.2AC36 channels. (D) Average density of active fusion channels as represented by the total current blocked by 2 mM ATP after patch excision. Numbers above the bars represent the number of patches considered. (E) Relative plasma membrane density of fusion proteins. Surface expression of HA-epitope tagged fusion proteins was estimated by a chemiluminescence detection technique.

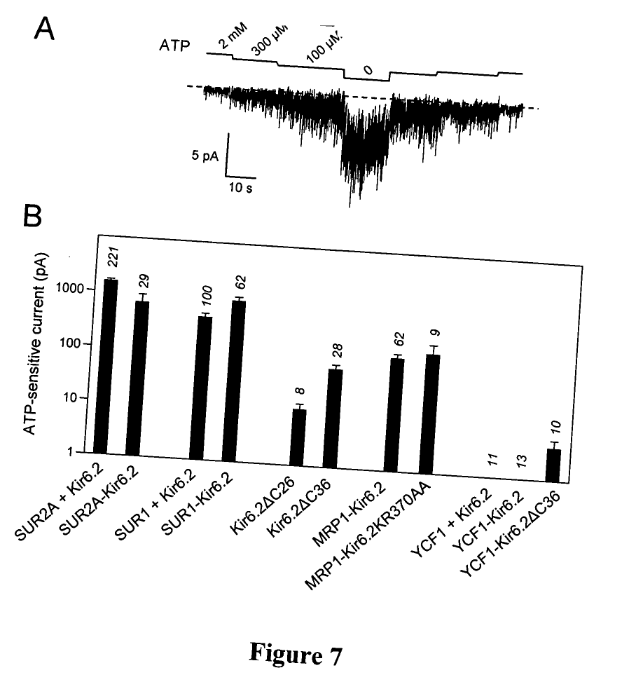

FIG. 7 illustrates the functional expression of YCF1-Kir6.2 fusion proteins lacking the ER retention. (A) Representative patch-clamp recording of YCF1-Kir6.2AC36 channels. (B) Average ATP-inhibitable currents after patch excision of the various fusion constructs. The numbers of patches considered are indicated within the graph. YCF1 coexpressed or fused with Kir6.2 produced no detectable exogenous current in all patches tested. Disruption of the RKR retention signal in the MRP1-Kir6.2 fusion produced no significant change in the number of active channels while this was required to observe YCF1-based fusion channels.

### EXAMPLE 1

### Materials and Methods

1) Molecular Biology

Standard PCR techniques were used to engineer fusion proteins with hexaglycine spacer, with or without added epitopes for immunodetection, and/or removed retention signals and/or added export signal, for optimal surface expression.

Mouse Kir6.2 (GenBank accession D50581), hamster SUR1 (GenBank accession L40623), rat SUR2A (GenBank accession D83598), yeast YCF1 (DDBJ/EMBL/GenBank accession No. L35237 ) and human MRP1 (DDBJ/EMBL/GenBank accession No. P33527) were subcloned in the Xenopus oocyte expression vectors derived from pGEMHE (Liman et al., Neuron, 1992, 9, 861-867). The resulting recombinant vectors are named: Kir6.2-pGEMHE, SUR1-pGEMHE, SUR2A-pGEMHE, YCF1-pGEMHE and MRP1-pGEMHE.

The tandem constructs were made by producing a fusion fragment containing the C-terminal end of the ABC protein cDNA and Kir6.2 using the Splicing by Overlap Extension (SOE) PCR technique (Horton et al., Gene, 1989, 77, 61-68).

To create the MRP1-Kir6.2 construct (SEQ ID NO: 1), in the first PCR reaction, MRP1 forward primer, 5′-GGT CAC CCA TGA GCT TCT TTG AGC G-3′ (SEQ ID NO: 13) and MRP1 fusion reverse primer, 5′-GCC TCC ACC TCC ACC TCC CAC CAA GCC GGC GTC-3′ (SEQ ID NO: 14) were used to generate a first junction fragment, using MRP1 as a template. In the second PCR reaction, Kir6.2 reverse primer, 5′-CAA GTG AGT GCT TAA GTG AGG AAC TGC AAC-3′ (SEQ ID NO: 15), and Kir6.2 fusion forward primer, 5′-GGA GGT GGA GGT GGA GGC ATG CTG TCC CGA AAG GGC-3′ (SEQ ID NO: 16), were used to create a second junction fragment, using Kir6.2 as a template. The two fragments were gel-purified and combined in a third overlap PCR reaction, using the MRP1 forward primer and the Kir6.2 reverse primer. The resulting 2.6 kb fragment was digested by ClaI and AflII restriction enzymes and ligated into identically cut MRP1-pGEMHE.

To create the SUR1-Kir6.2 construct, SUR1 forward primer 5′-CCG TGC CTT CAG GTA CGA GG-3′ (SEQ ID NO: 17) and SUR1 fusion reverse primer 5′-GCC TCC ACC TCC ACC TCC CTT GTC CGC ACG GAC AAA GG-3′ (SEQ ID NO: 18) were used for the first PCR reaction ; Kir6.2 reverse primer 5′-CAA GTG AGT GCT TAA GTG AGG AAC TGC AAC-3′ (SEQ ID NO: 15) and Kir6.2 fusion forward primer 5′-GGA GGT GGA GGT GGA GGC ATG CTG TCC CGA AAG GGC-3′ (SEQ ID NO: 16) were used for the second PCR reaction. The 2.3 kb fragment obtained after the third overlap PCR reaction was digested by NotI and AvrII restriction enzymes and ligated into identically cut SUR1-pGEMHE.

To create SUR2A-Kir6.2 construct (SEQ ID NO: 12), SUR2A forward primer 5′-TTC AAG CAA CGC ATG CTG GA-3′ (SEQ ID) NO: 19) and SUR2A fusion reverse primer 5′-GCC TCC ACC TCC ACC TCC CTT GTT GGT CAT CAC CAA-3′ (SEQ ID NO: 20) were used for the first PCR reaction; Kir6.2 reverse primer 5′-GTG CCC ACA CGC GTG AGT GGG GGC CCT AGG AAC TGC AAC TCA G-3′ (SEQ ID NO: 15) and Kir6.2 fusion forward primer 5′-GGA GGT GGA GGT GGA GGC ATG CTG TCC CGA AAG GGC-3′ (SEQ ID NO: 16) were used for the second PCR reaction. The 2.3 kb fragment obtained after the third overlap PCR reaction was digested by AgeI and MluI restriction enzymes and ligated into identically cut SUR2A-pGEMHE.

To create the YCF1-Kir6.2 construct (SEQ ID NO: 2), YCF1 forward primer 5′-CCC TCT ATC AAT GCT AAC CG-3′ (SEQ ID NO: 21) and YCF1 fusion reverse primer 5′-GCC TCC ACC TCC ACC TCC ATT TTC ATT GAC CAA ACC-3′ (SEQ ID NO: 22) were used for the first PCR reaction; Kir6.2 reverse primer 5′-GGG TGT TCT TGA GGC TGG-3′ (SEQ ID NO: 15) and Kir6.2 fusion forward primer 5′-GGA GGT GGA GGT GGA GGC ATG CTG TCC CGA AAG GGC-3′ (SEQ ID NO: 16) were used for the second PCR reaction. The 2.7 kb fragment obtained after the third overlap PCR reaction was digested by AflII and XbaI restriction enzymes and ligated into identically cut YCF1-pGEMHE.

Removal of the RKR endoplasmic reticulum retention signal of Kir6.2 was accomplished either by deletion of the last 26 or 36 residues of the C-terminal extremity (Tucker et al., Nature, 1997, 387: 179-183) through introduction of a premature stop codon at the correct position, or by site-directed mutagenesis of RKR to RAA (i.e., K370A and R371A). The resulting constructs, named Kir6.2Δ26, Kir6.2ΔC36, and Kir6.2KR370AA, were created by mutagenesis amplification (QuickChange site-directed mutagenesis kit, Stratagene). The sequences corresponding to the different constructs are as presented in Table 1: SUR2A-Kir6.2ΔC36 (SEQ ID NO: 3), MRP1-Kir6.2ΔC36 (SEQ ID NO: 4), YCF1-Kir6.2ΔC36 (SEQ ID NO: 5) and MRP1-Kir6.2[KR370AA] (SEQ ID NO: 6).

To generate HA-tagged constructs, an adaptation of the Quick Change Site Directed Mutagenesis Kit protocol (Stratagene) was used. By incorporating its coding sequence into PCR primers, an extracellular HA epitope (YPYDVPDYA, SEQ ID NO: 23) was introduced in Kir6.2 between amino acids P102 and G103. The sequences corresponding to the different constructs are as presented in Table 1: SUR2A-Kir6.2HA (SEQ ID NO: 7), MRP1-Kir6.2HA (SEQ ID NO: 8) and YCF1-Kir6.2ΔC36HA (SEQ ID NO: 9).

The sequence DLYAYMEKGIT (SEQ ID NO: 24) was additionnally inserted between G98 and D99, as described by Zerangue et al., Neuron, 1999, 22, 537-548

By using the same strategy, an export signal (FCYENE, SEQ ID NO: 25) was also introduced at the C-terminus of Kir6.2. The resulting construct, named YCF1-Kir6.2ΔC36HA-FCYENE, corresponds to SEQ ID NO: 10 (Table 1).

Plasmid DNAs were confirmed by restriction analysis and by sequencing of the PCR-derived regions. After linearization, mRNAs were produced in vitro using the T7 mMessage mMachine kit (Ambion).

mRNAs coding Kir6.2 (˜2 ng) and wild-type SURs (˜6 ng) or mRNAs coding the fusion constructs (˜8 ng) were co-injected into defoliculated Xenopus laevis oocytes. Injected oocytes were stored at 19° C. in Barth's solution (in mM: 1 KCl, 0.82 MgSO4, 88 NaCl, 2.4 NaHCO3, 0.41 CaCl2, 0.3 Ca(NO3)2, 16 HEPES, pH 7.4) with 100 U/ml penicillin, 100 μg/ml streptomycin, and 100 μg/ml gentamycin.

2) Oocyte Surface Protein Assays

Surface assay were performed at 4° C. 3-5 days after injection. For surface labelling, oocytes were blocked for 30 min in frog Ringer solution (ND96 : 96 mM NaCl, 2 mM KCl, 1 mM MgCl₂, 1.8 mM CaCl₂ and 5 mM Hepes supplemented with 2.5 mM sodium pyruvate, 50 mg/ml gentamicin and 0.6 mM theophyllin, pH 7.5) with 1% bovine serum albumin (BSA), labeled with 0.4 ng/μl rat monoclonal anti-HA antibody (3F10, Roche) in ND96-1% BSA for 30 min, washed in ND96-1% BSA, and incubated with 1 ng/μl horseradish peroxidase-coupled secondary antibody (HRP-conjugated goat anti rat F(Ab′)2 fragment, Jackson) in ND96-1% BSA for 30-40 min. Following a wash in ND96-1% BSA for 40 min, and in ND96 without BSA for 30 min, individual oocytes were placed in 50 [l SuperSignal ELISA Femto Sensitivity Substrate solution (Pierce), and incubated at room temperature for 25 s. Chemiluminescence was quantitated during 10 s reading in a Sirius Berthold luminometer. Constructs without HA epitope, H2O injected oocytes, and non-injected oocytes were used as negative controls.

3) Electrophysiology

Two to 15 days after injection, oocytes were devitellinized and recombinant K_{ATP} channels were characterized by the patch-clamp technique in the excised inside-out configuration (Hamill et al., Arch. Eur. J. Physiol., 1981, 391, 85-100). Patch pipettes (2-10 MΩ) contained 154 mM K⁺, 146 mM Cl⁻, 5 MM Mg²⁺, and 10 mM PIPES (pH 7.1). The cytoplasmic face of the patch was bathed in solutions which all contained 174 mM K⁺, 40 mM Cl⁻, 1 mM EGTA, 1 Mg²⁺, 10 mM PIPES (pH 7.1), and methanesulfonate (approximately, 160 mM, depending on the ATP and ADP amounts added—as the remaining anions. ATP and ADP (potassium salt; Sigma), SR47063 (20 mM stock in DMSO; Sanofi Recherche), diazoxide (100 mM stock in DMSO; Sigma) and P1075 (20 mM stock in DMSO; Leo Pharmaceutical Products) were added as specified. The membrane potential was maintained at −50 mV. Experiments were conducted at room temperature (22-24° C.).

Applications of the various solutions to the intracellular face of the patch was performed using a RSC-100 rapid-solution-changer (Bio-Logic). Analog signals were filtered at 300 Hz and sampled at 1 kHz. Slow fluctuations of the no-channel-open baseline of the signal were removed by interactive fitting of the baseline with a spline curve and subtraction of this fit from the signal. Acquisition, analysis, and presentation were performed with in-house software. Results are displayed as mean±s.e.m.

### EXAMPLE 2

### Functional Expression of SUR1-Kir6.2 and SUR2A-Kir6.2 Fusion Proteins

To assess the impact of subunit fusion on channel properties, SUR1-Kir6.2 and SUR2A-Kir6.2 fusion proteins were first constructed and the properties of the resulting channels were compared with those of the wild-type unfused channels. In these constructs, as well as all others used in this work, the C-terminal extremity of the ABC protein was covalently linked to the N-terminal extremity of Kir6.2 using a hexaglycine spacer.

The results obtained with SUR constructs show that the fusion proteins are correctly addressed to the plasma membrane and form channels that retain the basic regulatory properties of the wild-type channels ; they displayed a high sensitivity to ATP, activation by ADP, and activation by K channel openers (FIGS. 5A, 5B, 5C and 5D).

More precisely, as do wild-type cardiac K_{ATP} channels, in the presence of a partially-inhibiting ATP concentration (100 μM), SUR2A-Kir6.2 channels are activated by the K-channel-openers SR47063 and P1075 (FIG. 5A). SUR1-Kir6.2 channels are activated by diazoxide, a well-known opener of pancreatic K_{ATP} channels (FIG. 5B).

Since SUR normally acts as an ADP sensor, the effects of different ADP concentrations on the SUR1-Kir6.2 and SUR2A-Kir6.2 fusion channels was investigated and compared to those on wild-type channels as well as on Kir6.2 alone (FIG. 5C). It is known that ADP has dual effects on K_{ATP} channels due to its interaction with an activatory binding site of SUR (presumably NBD2) and with the inhibitory nucleotidic site of Kir6.2. Since activation occurs at lower concentrations than inhibition, the net effect of ADP at submillimolar concentrations on wild-type K_{ATP} channels is to increase open probability. At the same concentrations, ADP also causes activation of the fusion channels (FIG. 5C).

The truncation of the last 36 amino-acids containing the ER retention signal of Kir6.2 (Kir6.2ΔC36) generates channels that are correctly addressed to the plasma membrane when expressed alone. These SUR-less channels provide a means to assess the properties of the isolated Kir6.2 protein and estimate the role of the auxiliary SUR subunits. These channels are inhibited by ADP (FIG. 5C), confirming that, in the fusion proteins, SUR retains its ability to sense ADP and upregulate Kir6.2 gating accordingly.

### EXAMPLE 3

### Functional Expression of MRP1-Kir6.2 Fusion Proteins

The fusion of MRP1 and Kir6.2, injected in Xenopus oocytes, produces ATP-sensitive currents (FIG. 6A), demonstrating that MRP1-Kir6.2 forms functional channels in the plasma membrane. This result suggests that MRP1 does not interfere with the assembly and function of Kir6.2 tetramers and that it masks the Kir6.2 retention signal, allowing trafficking of the channel complex to the plasma membrane and delivery of functional artificial potassium ATP-sensitive channels. MRP1-Kir6.2 channels appear less sensitive to inhibition by ATP than either SUR-Kir6.2 fusions or wild-type channels (FIG. 6C). It should be noted however that there is a large variability in the nucleotide response of all channels tested in this study, in line with what has been long known for native KATP channels. Within the limits of this variability, the sensitivity to ATP of MRP1-Kir6.2 channels appears similar to that of Kir6.2ΔC36 channels (FIG. 6C).

MRP1 ability to sense intracellular ADP changes, like SUR does, was investigated next by evaluating the effect of ADP (100 μM and 300 μM) on MRP1-Kir6.2 channels. FIG. 6B shows that MRP1-Kir6.2 currents are reduced in a dose-dependent manner by addition of ADP, and, as a consequence, that the addition of MRP1 to Kir6.2 does not modify the sensitivity to ADP of Kir6.2 independently expressed.

The nucleotide responses of the MRP1 fusion channel was not affected by the presence of the Kir6.2 retention signal as demonstrated with the construct MRP1-Kir6.2KR370AA wherein the two last amino-acids of the RKR signal are converted to alanine residues; the MRP1-Kir6.2KR370AA construct displayed sensitivity to ATP and ADP that could not be distinguished from the non-mutated MRP1-Kir6.2 protein (FIGS. 6B and 6C).

In terms of surface expression, it appeared that currents in patches from oocytes expressing MRP1-Kir6.2 channels were 5- and 7-fold lower than measured with SUR2A-Kir6.2 and SUR1-Kir6.2, respectively (FIG. 6D). This could simply arise from genuine differences in expression levels which are influenced by a number of hard-to-control experimental factors such as oocyte variability and RNA quantity and quality. This could also reflect a reduced functionality of MRP1-Kir6.2 compared to SUR-Kir6.2, as would happen if most MRP1-Kir6.2 tetramers were inactive or if monomeric proteins were predominant. We examine this point by measuring total surface expression of the fusion proteins using chemiluminescent detection of a hemagglutinin epitope inserted in the first extracellular loop of Kir6.2. Results presented in FIG. 6E demonstrate that the surface density of MRP1-Kir6.2 proteins is 4- and 5-fold lower than that of SUR2A-Kir6.2 and SUR1-Kir6.2, respectively. Thus, as the lower level of current correlates well with a lower protein level it appears that the functional states of the MRP1-based and SUR-based tandems are equivalent.

Since no obvious difference between the activity of Kir6.2ΔC36 and of MRP1-Kir6.2 channels in response to ADP and ATP were observed, it may be speculated that MRP1, in contrast to SUR, cannot either sense or transmit to Kir6.2 internal nucleotides variations within the range of concentrations tested.

### EXAMPLE 4

### Functional Expression of YCF1-Kir6.2 Constructs Lacking the ER Retention Signal

If MRP1 fused to Kir6.2 can restore proper trafficking to the plasma membrane and form ATP-sensitive potassium channels, this effect might be unrelated to MRP1. Other membrane or even soluble proteins could do the same just by virtue of being attached to the N-terminal end of Kir6.2 and altering the cytoplasmic structure of the channel. The linker between the ABC protein and Kir6.2 might even be sufficient to affect trafficking itself.

As a control, to test the specificity of the shielding by MRP1 of the Kir6.2 retention signal, a fusion of Kir6.2 with an ABC protein highly homologous to MRP1, YCF1 (yeast cadmium transporter), was designed. MRP1 shares 56% homology with YCF1 and has been shown to complement YCF1 in yeast lacking the gene encoding this protein.

The results presented in FIG. 7B show that neither co-expression of YCF1 with Kir6.2, nor expression of the fusion YCF1-Kir6.2 produced detectable currents in all patches tested. Such negative results could be due to a number of reasons ranging from misfolding to incorrect addressing to impaired channel gating. One hypothesis for the absence of functional channels could be that YCF1 cannot properly shield the Kir6.2 retention signal. This was tested with the YCF1-Kir6.2ΔC36 construct wherein the last 36 residues of the YCF1-Kir6.2 construct were truncated to remove the RKR signal. Expression of YCF1-Kir6.2ΔC36 does produce ATP-sensitive currents (FIG. 7A) even if these currents are noticeably small compared to the other constructs (FIG. 7B). As removal of the retention signal in the YCF1-Kir6.2 construct augments the density of active channels in the plasma membrane, this is not the case for MRP1-Kir6.2 which yields similar current density with and without an intact retention signal (constructs MRP1-Kir6.2 and MRP1-Kir6.2KR370AA). These data are in agreement with MRP1, like SUR but unlike YCF1, being able to shield the retention signal of Kir6.2.

1. An hybrid protein consisting essentially of the fusion of a membrane protein with an ion channel which is not naturally coupled to said membrane protein.

2. The hybrid protein of claim 1, which comprises a spacer between the membrane protein and the ion channel.

3. The hybrid protein of claim 2, wherein said spacer consists of six glycine or ten glutamine residues.

4. The hybrid protein of claim 1, which comprises a tag, to facilitate the detection and/or the purification of said hybrid protein.

5. The hybrid protein of claim 1, wherein said membrane protein is a receptor.

6. The hybrid protein of claim 5, wherein said receptor is an hormone receptor.

7. The hybrid protein of claim 6, wherein said hormone receptor is the M2 muscarinic receptor.

8. The hybrid protein of claim 6, wherein said hormone receptor is the P2 adrenergic receptor.

9. The hybrid protein of claim 5, wherein said receptor is a receptor for a pollutant/contaminant.

10. The hybrid protein of claim 5, wherein said receptor is an olfactive receptor.

11. The hybrid protein of claim 1, wherein said membrane protein is a transporter.

12. The hybrid protein of claim 11, wherein said transporter is an ABC transporter.

13. The hybrid protein of claim 12, wherein said ABC transporter is from the MRP class.

14. The hybrid protein of claim 13, wherein said ABC transporter is CFTR.

15. The hybrid protein of claim 13, wherein said ABC transporter is MRP1.

16. The hybrid protein of claim 13, wherein said ABC transporter is YCF1.

17. The hybrid protein of claim 13, wherein said ABC transporter is SUR.

18. The hybrid protein of claim 12, wherein said ABC transporter is Mdr1.

19. The hybrid protein of claim 11, wherein said transporter is a transporter for a pollutant/contaminant.

20. The hybrid protein of claim 19, wherein said transporter is an heavy metal transporter.

21. The hybrid protein of claim 1, wherein said ion channel is a potassium channel.

22. The hybrid protein of claim 21, wherein said potassium channel is an ATP-sensitive potassium channel.

23. The hybrid protein of claim 22, wherein said ATP-sensitive potassium channel is from the Kir family.

24. The hybrid protein of claim 23, wherein said ATP-sensitive potassium channel is Kir6.2.

25. The hybrid protein of claim 24, which is SEQ ID NO: 1 to 11.

26. The hybrid protein of claim 1, wherein said ion channel is a voltage dependent channel.

27. The hybrid protein of claim 26, wherein said voltage dependent channel is from the Kv family.

28. The hybrid protein of claim 1, wherein said ion channel is a mechanosensitive channel.

29. The hybrid protein of claim 28, wherein said mechanosensitive channel is MscL.

30. A polynucleotide encoding the hybrid protein of claim 1.

31. A polynucleotide encoding the hybrid protein of claim 25.

32. A primer able to amplify the polynucleotide of claim 25, which is SEQ ID NO: 13 to 16 and 21, 22.

33. A recombinant vector comprising the polynucleotide of claim 30.

34. An host cell expressing the hybrid protein of claim 1.

35. An electrical sensor comprising the hybrid protein of claim 1, incorporated in a membrane.

36. A method for the screening of an agonist of a membrane protein, comprising the step of:
bringing a drug to test in contact with the electrical sensor of claim 35,
measuring the resulting electrical signal by appropriate means, and
selecting the drugs which induce an electrical signal.

37. A method for the screening of an antagonist of a membrane protein, comprising the step of:
bringing a drug to test in contact with the electrical sensor of claim 35, and with a ligand/substrate of said membrane protein,
measuring the resulting electrical signal by appropriate means, and
selecting the drugs which block the electrical signal induced by said ligand/substrate.

38. The method of claim 36, wherein said electrical sensor comprises an hybrid protein according to claim 15, to screen anticancer drugs or multidrug reversing agents.

39. The method of claim 37, wherein said electrical sensor comprises an hybrid protein according to claim 15, to screen anticancer drugs or multidrug reversing agents

40. The method of claim 36, wherein said electrical sensor comprises an hybrid protein according to claim 17, to screen antidiabetic, antiischemic or antihypertensive drugs.

41. The method of claim 37, wherein said electrical sensor comprises an hybrid protein according to claim 17, to screen antidiabetic, antiischemic or antihypertensive drugs.

42. A method for the detection of a contaminant/pollutant, comprising the step of:
bringing a sample to be tested in contact with the electrical sensor of claim 35,
measuring the resulting electrical signal by appropriate means, and
detecting the presence of said contaminant/pollutant in said sample.

43. The method of claim 42, wherein said electrical sensor comprises an hybrid protein according to claim 16, to detect heavy metals such as nickel, cadmium, arsenite and mercury.

44. A method for assaying the activity of membrane protein, comprising the step of:
bringing a ligand/substrate of said membrane protein in contact with the electrical sensor of claim 35, and
measuring the resulting electrical signal by appropriate means.

45. A kit for the screening of an agonist/antagonist of a membrane protein comprising at least the electrical sensor of claim 35.

46. A kit for the detection of a contaminant/pollutant comprising at least the electrical sensor of claim 35.


SEQUENCE LISTING (25 sequences)

SEQ ID NO: 1 - protein, 1927 aa, Homo sapiens
MALRGFCSADGSDPLWDWNVTWNTSNPDFTKCFQNTVLVWVPCFYLWACFPFYFLYLSRHDRGYIQMTPLNKTKTALGFLLWIVCWADLFYSFWERSRGIFLAPVFLVSPTLLGITTLLATFLIQLERRKGVQSSGIMLTFWLVALVCALAILRSKIMTALKEDAQVDLFRDITFYVYFSLLLIQLVLSCFSDRSPLFSETIHDPNPCPESSASFLSRITFWWITGLIVRGYRQPLEGSDLWSLNKEDTSEQVVPVLVKNWKKECAKTRKQPVKVVYSSKDPAQPKESSKVDANEEVEALIVKSPQKEWNPSLFKVLYKTFGPYFLMSFFFKAIHDLMMFSGPQILKLLIKFVNDTKAPDWQGYFYTVLLFVTACLQTLVLHQYFHICFVSGMRIKTAVIGAVYRKALVITNSARKSSTVGEIVNLMSVDAQRFMDLATYINMIWSAPLQVILALYLLWLNLGPSVLAGVAVMVLMVPVNAVMAMKTKTYQVAHMKSKDNRIKLMNEILNGIKVLKLYAWELAFKDKVLAIRQEELKVLKKSAYLSAVGTFTWVCTPFLVALCTFAVYVTIDENNILDAQTAFVSLALFNILRFPLNILPMVISSIVQASVSLKRLRIFLSHEELEPDSIERRPVKDGGGTNSITVRNATFTWARSDPPTLNGITFSIPEGALVAVVGQVGCGKSSLLSALLAEMDKVEGHVAIKGSVAYVPQQAWIQNDSLRENILFGCQLEEPYYRSVIQACALLPDLEILPSGDRTEIGEKGVNLSGGQKQRVSLARAVYSNADIYLFDDPLSAVDAHVGKHIFENVIGPKGMLKNKTRILVTHSMSYLPQVDVIIVMSGGKISEMGSYQELLARDGAFAEFLRTYASTEQEQDAEENGVTGVSGPGKEAKQMENGMLVTDSAGKQLQRQLSSSSSYSGDISRHHNSTAELQKAEAKKEETWKLMEADKAQTGQVKLSVYWDYMKAIGLFISFLSIFLFMCNHVSALASNYWLSLWTDDPIVNGTQEHTKVRLSVYGALGISQGIAVFGYSMAVSIGGILASRCLHVDLLHSILRSPMSFFERTPSGNLVNRFSKELDTVDSMIPEVIKMFMGSLFNVIGACIVILLATPIAAIIIPPLGLIYFFVQRFYVASSRQLKRLESVSRSPVYSHFNETLLGVSVIRAFEEQERFIHQSDLKVDENQKAYYPSIVANRWLAVRLECVGNCIVLFAALFAVISRHSLSAGLVGLSVSYSLQVTTYLNWLVRMSSEMETNIVAVERLKEYSETEKEAPWQIQETAPPSSWPQVGRVEFRNYCLRYREDLDFVLRHINVTINGGEKVGIVGRTGAGKSSLTLGLFRINESAEGEIIIDGINIAKIGLHDLRFKITIIPQDPVLFSGSLRMNLDPFSQYSDEEVWTSLELAHLKDFVSALPDKLDHECAEGGENLSVGQRQLVCLARALLRKTKILVLDEATAAVDLETDDLIQSTIRTQFEDCTVLTIAHRLNTIMDYTRVIVLDKGEIQEYGAPSDLLQQRGLFYSMAKDAGLVGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLAPGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRSLLDALTLASSRGPLRKRSVAVAKAKPKFSISPDSLS

SEQ ID NO: 2 - protein, 1911 aa, Saccharomyces cerevisiae
MAGNLVSWACKLCRSPEGFGPISFYGDFTQCFIDGVILNLSAIFMITFGIRDLVNLCKKKHSGIKYRRNWIIVSRMALVLLEIAFVSLASLNISKEEAENFTIVSQYASTMLSLFVALALHWIEYDRSVVANTVLLFYWLFETFGNFAKLINILIRHTYEGIWYSGQTGFILTLFQVITCASILLLEALPKKPLMPHQHIHQTLTRRKPNPYDSANIFSRITFSWMSGLMKTGYEKYLVEADLYKLPRNFSSEELSQKLEKNWENELKQKSNPSLSWAICRTFGSKMLLAAFFKAIHDVLAFTQPQLLRILIKFVTDYNSERQDDHSSLQGFENNHPQKLPIVRGFLIAFAMFLVGFTQTSVLHQYFLNVFNTGMYIKSALTALIYQKSLVLSNEASGLSSTGDIVNLMSVDVQKLQDLTQWLNLIWSGPFQIIICLYSLYKLLGNSMWVGVIILVIMMPLNSFLMRIQKKLQKSQMKYKDERTRVISEILNNIKSLKLYAWEKPYREKLEEVRNNKELKNLTKLGCYMAVTSFQFNIVPFLVSCCTFAVFVYTEDRALTTDLVFPALTLFNLLSFPLMIIPMVLNSFIEASVSIGRLFTFFTNEELQPDSVQRLPKVKNIGDVAINIGDDATFLWQRKPEYKVALKNINFQAKKGNLTCIVGKVGSGKTALLSCMLGDRFRVKGFATVHGSVAYVSQVPWIMNGTVKENILFGHRYDAEFYEKTIKACALTIDLAILMDGDKTLVGEKGISLSGGQKARLSLARAVYARADTYLLDDPLAAVDEHVARHLIEHVLGPNGLLHTKTKVLATNKVSALSIADSIALLDNGEITQQGTYDEITKDADSPLWKLLNNYGKKNNGKSNEFGDSSESSVRESSIPVEGELEQLQKLNDLDFGNSDAISLRRASDATLGSIDFGDDENIAKREHREQGKVKWNIYLEYAKACNPKSVCVFILFIVISMFLSVMGNVWLKHWSEVNSRYGSNPNAARYLAIYFALGIGSALATLIQTIVLWVFCTIHASKYLHNLMTNSVLRAPMTFFETTPIGRILNRFSNDIYKVDALLGRTFSQFFVNAVKVTFTITVICATTWQFIFIIIPLSVFYIYYQQYYLRTSRELRRLDSITRSPIYSHFQETLGGLATVRGYSQQKRFSHINQCRIDNNMSAFYPSINANRWLAYRLELIGSIIILGAATLSVFRLKQGTLTAGMVGLSLSYALQITQTLNWIVRMTVEVETNIVSVERIKEYADLKSEAPLIVEGHRPPKEWPSQGDIKFNNYSTRYRPELDLVLKHINIHIKPNEKVGIVGRTGAGKSSLTLALFRMIEASEGNIVIDNIAINEIGLYDLRHKLSIIPQDSQVFEGTVRENIDPINQYTDEAIWRALELSHLKEHVLSMSNDGLDAQLTEGGGNLSVGQRQLLCLARAMLVPSKILVLDEATAAVDVETDKVVQETIRTAFKDRTILTIAHRLNTIMDSDRIIVLDNGKVAEFDSPGQLLSDNKSLFYSLCMEAGLVNENGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLAPGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRSLLDALTLASSRGPLRKRSVAVAKAKPKFSISPDSLS

SEQ ID NO: 3 - protein, 1905 aa, Rattus rattus
MSLSFCGNNISSYNIYHGVLQNPCFVDALNLVPHVFLLFITFPILFIGWGSQSSKVQIHHNTWLHFPGHNLRWILTFALLFVHVCEIAEGIVSDSQRASRHLHLFMPAVMGFVATTTSIVYYHNIETSNFPKLLLALFLYWVMAFITKTIKLVKYWQLGWGMSDLRFCITGVMVILNGLLMAVEINVIRVRRYVFFMNPQKVKPPEDLQDLGVRFLQPFVNLLSKATYWWMNTLIISAHRKPIDLKAIGKLPIAMRAVTNYVCLKEAYEEQKKKAADHPNRTPSIWLAMYRAFGRPILLSSTFRYLADLLGFAGPLCISGIVQRVNEPKNNTTRFSETLSSKEFLENAHVLAVLLFLALILQRTFLQASYYVTIETGINLRGALLAMIYNKILRLSTSNLSMGEMTLGQINNLVAIETNQLMWFLFLCPNLWAMPVQIIMGVILLYNLLGSSALVGAAVIVLLAPIQYFIATKLAEAQKSTLDYSTERLKKTNEILKGIKLLKLYAWEHIFCKSVEETRMKELSSLKTFALYTSLSIFMNAAIPIAAVLATFVTHAYASGNNLKPAEAFASLSLFHILVTPLFLLSTVVRFAVKAIISVQKLNEFLLSDEIGEDSWRTGEGTLPFESCKKHTGVQSKPINRKQPGRYHLDNYEQARRLRPAETEDVAIKVTNGYFSWGSGLATLSNIDIRIPTGQLTMIVGQVGCGKSSLLLAILGEMQTLEGKVYWNNVNESEPSFEATRSRSRYSVAYAAQKPWLLNATVEENITFGSSFNRQRYKAVTDACSLQPDIDLLPFGDQTEIGERGINLSGGQRQRICVARALYQNTNIVFLDDPFSALDIHLSDHLMQEGILKFLQDDKRTVVLVTHKLQYLTHADWIIAMKDGSVLREGTLKDIQTKDVELYEHWKTLMNRQDQELEKDMEADQTTLERKTLRRAMYSREAKAQMEDEDEEEEEEEDEDDNMSTVMRLRTKMPWKTCWWYLTSGGFFLLFLMIFSKLLKHSVIVAIDYWLATWTSEYSINDPGKADQTFYVAGFSILCGAGIFLCLVTSLTVEWMGLTAAKNLHHNLLNKIILGPIRFFDTTPLGLILNRFSADTNIIDQHIPPTLESLTRSTLLCLSAIGMISYATPVFLIALAPLGVAFYFIQKYFRVASKDLQELDDSTQLPLLCHFSETAEGLTTIRAFRHETRFKQRMLELTDTNNIAYLFLSAANRWLEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDATVTEGGENFSVGQRQLFCLARAFVRKSSILIMDEATASIDMATENILQKVVMTAFADRTVVTIAHRVSSIMDAGLVLVFSEGILVECDTGPNLLQHKNGLFSTLVMTNKGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLAPGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRS

SEQ ID NO: 4 - protein, 1891 aa, Homo sapiens
MALRGFCSADGSDPLWDWNVTWNTSNPDFTKCFQNTVLVWVPCFYLWACFPFYFLYLSRHDRGYIQMTPLNKTKTALGFLLWIVCWADLFYSFWERSRGIFLAPVFLVSPTLLGITTLLATFLIQLERRKGVQSSGIMLTFWLVALVCALAILRSKIMTALKEDAQVDLFRDITFYVYFSLLLIQLVLSCFSDRSPLFSETIHDPNPCPESSASFLSRITFWWITGLIVRGYRQPLEGSDLWSLNKEDTSEQVVPVLVKNWKKECAKTRKQPVKVVYSSKDPAQPKESSKVDANEEVEALIVKSPQKEWNPSLFKVLYKTFGPYFLMSFFFKAIHDLMMFSGPQILKLLIKFVNDTKAPDWQGYFYTVLLFVTACLQTLVLHQYFHICFVSGMRIKTAVIGAVYRKALVITNSARKSSTVGEIVNLMSVDAQRFMDLATYINMIWSAPLQVILALYLLWLNLGPSVLAGVAVMVLMVPVNAVMAMKTKTYQVAHMKSKDNRIKLMNEILNGIKVLKLYAWELAFKDKVLAIRQEELKVLKKSAYLSAVGTFTWVCTPFLVALCTFAVYVTIDENNILDAQTAFVSLALFNILRFPLNILPMVISSIVQASVSLKRLRIFLSHEELEPDSIERRPVKDGGGTNSITVRNATFTWARSDPPTLNGITFSIPEGALVAVVGQVGCGKSSLLSALLAEMDKVEGHVAIKGSVAYVPQQAWIQNDSLRENILFGCQLEEPYYRSVIQACALLPDLEILPSGDRTEIGEKGVNLSGGQKQRVSLARAVYSNADIYLFDDPLSAVDAHVGKHIFENVIGPKGMLKNKTRILVTHSMSYLPQVDVIIVMSGGKISEMGSYQELLARDGAFAEFLRTYASTEQEQDAEENGVTGVSGPGKEAKQMENGMLVTDSAGKQLQRQLSSSSSYSGDISRHHNSTAELQKAEAKKEETWKLMEADKAQTGQVKLSVYWDYMKAIGLFISFLSIFLFMCNHVSALASNYWLSLWTDDPIVNGTQEHTKVRLSVYGALGISQGIAVFGYSMAVSIGGILASRCLHVDLLHSILRSPMSFFERTPSGNLVNRFSKELDTVDSMIPEVIKMFMGSLFNVIGACIVILLATPIAAIIIPPLGLIYFFVQRFYVASSRQLKRLESVSRSPVYSHFNETLLGVSVIRAFEEQERFIHQSDLKVDENQKAYYPSIVANRWLAVRLECVGNCIVLFAALFAVISRHSLSAGLVGLSVSYSLQVTTYLNWLVRMSSEMETNIVAVERLKEYSETEKEAPWQIQETAPPSSWPQVGRVEFRNYCLRYREDLDFVLRHINVTINGGEKVGIVGRTGAGKSSLTLGLFRINESAEGEIIIDGINIAKIGLHDLRFKITIIPQDPVLFSGSLRMNLDPFSQYSDEEVWTSLELAHLKDFVSALPDKLDHECAEGGENLSVGQRQLVCLARALLRKTKILVLDEATAAVDLETDDLIQSTIRTQFEDCTVLTIAHRLNTIMDYTRVIVLDKGEIQEYGAPSDLLQQRGLFYSMAKDAGLVGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLAPGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRS

SEQ ID NO: 5 - protein, 1875 aa, Saccharomyces cerevisiae
MAGNLVSWACKLCRSPEGFGPISFYGDFTQCFIDGVILNLSAIFMITFGIRDLVNLCKKKHSGIKYRRNWIIVSRMALVLLEIAFVSLASLNISKEEAENFTIVSQYASTMLSLFVALALHWIEYDRSVVANTVLLFYWLFETFGNFAKLINILIRHTYEGIWYSGQTGFILTLFQVITCASILLLEALPKKPLMPHQHIHQTLTRRKPNPYDSANIFSRITFSWMSGLMKTGYEKYLVEADLYKLPRNFSSEELSQKLEKNWENELKQKSNPSLSWAICRTFGSKMLLAAFFKAIHDVLAFTQPQLLRILIKFVTDYNSERQDDHSSLQGFENNHPQKLPIVRGFLIAFAMFLVGFTQTSVLHQYFLNVFNTGMYIKSALTALIYQKSLVLSNEASGLSSTGDIVNLMSVDVQKLQDLTQWLNLIWSGPFQIIICLYSLYKLLGNSMWVGVIILVIMMPLNSFLMRIQKKLQKSQMKYKDERTRVISEILNNIKSLKLYAWEKPYREKLEEVRNNKELKNLTKLGCYMAVTSFQFNIVPFLVSCCTFAVFVYTEDRALTTDLVFPALTLFNLLSFPLMIIPMVLNSFIEASVSIGRLFTFFTNEELQPDSVQRLPKVKNIGDVAINIGDDATFLWQRKPEYKVALKNINFQAKKGNLTCIVGKVGSGKTALLSCMLGDRFRVKGFATVHGSVAYVSQVPWIMNGTVKENILFGHRYDAEFYEKTIKACALTIDLAILMDGDKTLVGEKGISLSGGQKARLSLARAVYARADTYLLDDPLAAVDEHVARHLIEHVLGPNGLLHTKTKVLATNKVSALSIADSIALLDNGEITQQGTYDEITKDADSPLWKLLNNYGKKNNGKSNEFGDSSESSVRESSIPVEGELEQLQKLNDLDFGNSDAISLRRASDATLGSIDFGDDENIAKREHREQGKVKWNIYLEYAKACNPKSVCVFILFIVISMFLSVMGNVWLKHWSEVNSRYGSNPNAARYLAIYFALGIGSALATLIQTIVLWVFCTIHASKYLHNLMTNSVLRAPMTFFETTPIGRILNRFSNDIYKVDALLGRTFSQFFVNAVKVTFTITVICATTWQFIFIIIPLSVFYIYYQQYYLRTSRELRRLDSITRSPIYSHFQETLGGLATVRGYSQQKRFSHINQCRIDNNMSAFYPSINANRWLAYRLELIGSIIILGAATLSVFRLKQGTLTAGMVGLSLSYALQITQTLNWIVRMTVEVETNIVSVERIKEYADLKSEAPLIVEGHRPPKEWPSQGDIKFNNYSTRYRPELDLVLKHINIHIKPNEKVGIVGRTGAGKSSLTLALFRMIEASEGNIVIDNIAINEIGLYDLRHKLSIIPQDSQVFEGTVRENIDPINQYTDEAIWRALELSHLKEHVLSMSNDGLDAQLTEGGGNLSVGQRQLLCLARAMLVPSKILVLDEATAAVDVETDKVVQETIRTAFKDRTILTIAHRLNTIMDSDRIIVLDNGKVAEFDSPGQLLSDNKSLFYSLCMEAGLVNENGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLAPGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRS

SEQ ID NO: 6 - protein, 1927 aa, Homo sapiens
MALRGFCSADGSDPLWDWNVTWNTSNPDFTKCFQNTVLVWVPCFYLWACFPFYFLYLSRHDRGYIQMTPLNKTKTALGFLLWIVCWADLFYSFWERSRGIFLAPVFLVSPTLLGITTLLATFLIQLERRKGVQSSGIMLTFWLVALVCALAILRSKIMTALKEDAQVDLFRDITFYVYFSLLLIQLVLSCFSDRSPLFSETIHDPNPCPESSASFLSRITFWWITGLIVRGYRQPLEGSDLWSLNKEDTSEQVVPVLVKNWKKECAKTRKQPVKVVYSSKDPAQPKESSKVDANEEVEALIVKSPQKEWNPSLFKVLYKTFGPYFLMSFFFKAIHDLMMFSGPQILKLLIKFVNDTKAPDWQGYFYTVLLFVTACLQTLVLHQYFHICFVSGMRIKTAVIGAVYRKALVITNSARKSSTVGEIVNLMSVDAQRFMDLATYINMIWSAPLQVILALYLLWLNLGPSVLAGVAVMVLMVPVNAVMAMKTKTYQVAHMKSKDNRIKLMNEILNGIKVLKLYAWELAFKDKVLAIRQEELKVLKKSAYLSAVGTFTWVCTPFLVALCTFAVYVTIDENNILDAQTAFVSLALFNILRFPLNILPMVISSIVQASVSLKRLRIFLSHEELEPDSIERRPVKDGGGTNSITVRNATFTWARSDPPTLNGITFSIPEGALVAVVGQVGCGKSSLLSALLAEMDKVEGHVAIKGSVAYVPQQAWIQNDSLRENILFGCQLEEPYYRSVIQACALLPDLEILPSGDRTEIGEKGVNLSGGQKQRVSLARAVYSNADIYLFDDPLSAVDAHVGKHIFENVIGPKGMLKNKTRILVTHSMSYLPQVDVIIVMSGGKISEMGSYQELLARDGAFAEFLRTYASTEQEQDAEENGVTGVSGPGKEAKQMENGMLVTDSAGKQLQRQLSSSSSYSGDISRHHNSTAELQKAEAKKEETWKLMEADKAQTGQVKLSVYWDYMKAIGLFISFLSIFLFMCNHVSALASNYWLSLWTDDPIVNGTQEHTKVRLSVYGALGISQGIAVFGYSMAVSIGGILASRCLHVDLLHSILRSPMSFFERTPSGNLVNRFSKELDTVDSMIPEVIKMFMGSLFNVIGACIVILLATPIAAIIIPPLGLIYFFVQRFYVASSRQLKRLESVSRSPVYSHFNETLLGVSVIRAFEEQERFIHQSDLKVDENQKAYYPSIVANRWLAVRLECVGNCIVLFAALFAVISRHSLSAGLVGLSVSYSLQVTTYLNWLVRMSSEMETNIVAVERLKEYSETEKEAPWQIQETAPPSSWPQVGRVEFRNYCLRYREDLDFVLRHINVTINGGEKVGIVGRTGAGKSSLTLGLFRINESAEGEIIIDGINIAKIGLHDLRFKITIIPQDPVLFSGSLRMNLDPFSQYSDEEVWTSLELAHLKDFVSALPDKLDHECAEGGENLSVGQRQLVCLARALLRKTKILVLDEATAAVDLETDDLIQSTIRTQFEDCTVLTIAHRLNTIMDYTRVIVLDKGEIQEYGAPSDLLQQRGLFYSMAKDAGLVGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLAPGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRSLLDALTLASSRGPLRAASVAVAKAKPKFSISPDSLS

SEQ ID NO: 7 - protein, 1961 aa, Rattus rattus
MSLSFCGNNISSYNIYHGVLQNPCFVDALNLVPHVFLLFITFPILFIGWGSQSSKVQIHHNTWLHFPGHNLRWILTFALLFVHVCEIAEGIVSDSQRASRHLHLFMPAVMGFVATTTSIVYYHNIETSNFPKLLLALFLYWVMAFITKTIKLVKYWQLGWGMSDLRFCITGVMVILNGLLMAVEINVIRVRRYVFFMNPQKVKPPEDLQDLGVRFLQPFVNLLSKATYWWMNTLIISAHRKPIDLKAIGKLPIAMRAVTNYVCLKEAYEEQKKKAADHPNRTPSIWLAMYRAFGRPILLSSTFRYLADLLGFAGPLCISGIVQRVNEPKNNTTRFSETLSSKEFLENAHVLAVLLFLALILQRTFLQASYYVTIETGINLRGALLAMIYNKILRLSTSNLSMGEMTLGQINNLVAIETNQLMWFLFLCPNLWAMPVQIIMGVILLYNLLGSSALVGAAVIVLLAPIQYFIATKLAEAQKSTLDYSTERLKKTNEILKGIKLLKLYAWEHIFCKSVEETRMKELSSLKTFALYTSLSIFMNAAIPIAAVLATFVTHAYASGNNLKPAEAFASLSLFHILVTPLFLLSTVVRFAVKAIISVQKLNEFLLSDEIGEDSWRTGEGTLPFESCKKHTGVQSKPINRKQPGRYHLDNYEQARRLRPAETEDVAIKVTNGYFSWGSGLATLSNIDIRIPTGQLTMIVGQVGCGKSSLLLAILGEMQTLEGKVYWNNVNESEPSFEATRSRSRYSVAYAAQKPWLLNATVEENITFGSSFNRQRYKAVTDACSLQPDIDLLPFGDQTEIGERGINLSGGQRQRICVARALYQNTNIVFLDDPFSALDIHLSDHLMQEGILKFLQDDKRTVVLVTHKLQYLTHADWIIAMKDGSVLREGTLKDIQTKDVELYEHWKTLMNRQDQELEKDMEADQTTLERKTLRRAMYSREAKAQMEDEDEEEEEEEDEDDNMSTVMRLRTKMPWKTCWWYLTSGGFFLLFLMIFSKLLKHSVIVAIDYWLATWTSEYSINDPGKADQTFYVAGFSILCGAGIFLCLVTSLTVEWMGLTAAKNLHHNLLNKIILGPIRFFDTTPLGLILNRFSADTNIIDQHIPPTLESLTRSTLLCLSAIGMISYATPVFLIALAPLGVAFYFIQKYFRVASKDLQELDDSTQLPLLCHFSETAEGLTTIRAFRHETRFKQRMLELTDTNNIAYLFLSAANRWLEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDATVTEGGENFSVGQRQLFCLARAFVRKSSILIMDEATASIDMATENILQKVVMTAFADRTVVTIAHRVSSIMDAGLVLVFSEGILVECDTGPNLLQHKNGLFSTLVMTNKGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLYAYMEKGITDLAPYPYDVPDYAGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRSLLDALTLASSRGPLRKRSVAVAKAKPKFSISPDSLS

SEQ ID NO: 8 - protein, 1947 aa, Homo sapiens
MALRGFCSADGSDPLWDWNVTWNTSNPDFTKCFQNTVLVWVPCFYLWACFPFYFLYLSRHDRGYIQMTPLNKTKTALGFLLWIVCWADLFYSFWERSRGIFLAPVFLVSPTLLGITTLLATFLIQLERRKGVQSSGIMLTFWLVALVCALAILRSKIMTALKEDAQVDLFRDITFYVYFSLLLIQLVLSCFSDRSPLFSETIHDPNPCPESSASFLSRITFWWITGLIVRGYRQPLEGSDLWSLNKEDTSEQVVPVLVKNWKKECAKTRKQPVKVVYSSKDPAQPKESSKVDANEEVEALIVKSPQKEWNPSLFKVLYKTFGPYFLMSFFFKAIHDLMMFSGPQILKLLIKFVNDTKAPDWQGYFYTVLLFVTACLQTLVLHQYFHICFVSGMRIKTAVIGAVYRKALVITNSARKSSTVGEIVNLMSVDAQRFMDLATYINMIWSAPLQVILALYLLWLNLGPSVLAGVAVMVLMVPVNAVMAMKTKTYQVAHMKSKDNRIKLMNEILNGIKVLKLYAWELAFKDKVLAIRQEELKVLKKSAYLSAVGTFTWVCTPFLVALCTFAVYVTIDENNILDAQTAFVSLALFNILRFPLNILPMVISSIVQASVSLKRLRIFLSHEELEPDSIERRPVKDGGGTNSITVRNATFTWARSDPPTLNGITFSIPEGALVAVVGQVGCGKSSLLSALLAEMDKVEGHVAIKGSVAYVPQQAWIQNDSLRENILFGCQLEEPYYRSVIQACALLPDLEILPSGDRTEIGEKGVNLSGGQKQRVSLARAVYSNADIYLFDDPLSAVDAHVGKHIFENVIGPKGMLKNKTRILVTHSMSYLPQVDVIIVMSGGKISEMGSYQELLARDGAFAEFLRTYASTEQEQDAEENGVTGVSGPGKEAKQMENGMLVTDSAGKQLQRQLSSSSSYSGDISRHHNSTAELQKAEAKKEETWKLMEADKAQTGQVKLSVYWDYMKAIGLFISFLSIFLFMCNHVSALASNYWLSLWTDDPIVNGTQEHTKVRLSVYGALGISQGIAVFGYSMAVSIGGILASRCLHVDLLHSILRSPMSFFERTPSGNLVNRFSKELDTVDSMIPEVIKMFMGSLFNVIGACIVILLATPIAAIIIPPLGLIYFFVQRFYVASSRQLKRLESVSRSPVYSHFNETLLGVSVIRAFEEQERFIHQSDLKVDENQKAYYPSIVANRWLAVRLECVGNCIVLFAALFAVISRHSLSAGLVGLSVSYSLQVTTYLNWLVRMSSEMETNIVAVERLKEYSETEKEAPWQIQETAPPSSWPQVGRVEFRNYCLRYREDLDFVLRHINVTINGGEKVGIVGRTGAGKSSLTLGLFRINESAEGEIIIDGINIAKIGLHDLRFKITIIPQDPVLFSGSLRMNLDPFSQYSDEEVWTSLELAHLKDFVSALPDKLDHECAEGGENLSVGQRQLVCLARALLRKTKILVLDEATAAVDLETDDLIQSTIRTQFEDCTVLTIAHRLNTIMDYTRVIVLDKGEIQEYGAPSDLLQQRGLFYSMAKDAGLVGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLYAYMEKGITDLAPYPYDVPDYAGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRSLLDALTLASSRGPLRKRSVAVAKAKPKFSISPDSLS

SEQ ID NO: 9 - protein, 1895 aa, Saccharomyces cerevisiae
MAGNLVSWACKLCRSPEGFGPISFYGDFTQCFIDGVILNLSAIFMITFGIRDLVNLCKKKHSGIKYRRNWIIVSRMALVLLEIAFVSLASLNISKEEAENFTIVSQYASTMLSLFVALALHWIEYDRSVVANTVLLFYWLFETFGNFAKLINILIRHTYEGIWYSGQTGFILTLFQVITCASILLLEALPKKPLMPHQHIHQTLTRRKPNPYDSANIFSRITFSWMSGLMKTGYEKYLVEADLYKLPRNFSSEELSQKLEKNWENELKQKSNPSLSWAICRTFGSKMLLAAFFKAIHDVLAFTQPQLLRILIKFVTDYNSERQDDHSSLQGFENNHPQKLPIVRGFLIAFAMFLVGFTQTSVLHQYFLNVFNTGMYIKSALTALIYQKSLVLSNEASGLSSTGDIVNLMSVDVQKLQDLTQWLNLIWSGPFQIIICLYSLYKLLGNSMWVGVIILVIMMPLNSFLMRIQKKLQKSQMKYKDERTRVISEILNNIKSLKLYAWEKPYREKLEEVRNNKELKNLTKLGCYMAVTSFQFNIVPFLVSCCTFAVFVYTEDRALTTDLVFPALTLFNLLSFPLMIIPMVLNSFIEASVSIGRLFTFFTNEELQPDSVQRLPKVKNIGDVAINIGDDATFLWQRKPEYKVALKNINFQAKKGNLTCIVGKVGSGKTALLSCMLGDRFRVKGFATVHGSVAYVSQVPWIMNGTVKENILFGHRYDAEFYEKTIKACALTIDLAILMDGDKTLVGEKGISLSGGQKARLSLARAVYARADTYLLDDPLAAVDEHVARHLIEHVLGPNGLLHTKTKVLATNKVSALSIADSIALLDNGEITQQGTYDEITKDADSPLWKLLNNYGKKNNGKSNEFGDSSESSVRESSIPVEGELEQLQKLNDLDFGNSDAISLRRASDATLGSIDFGDDENIAKREHREQGKVKWNIYLEYAKACNPKSVCVFILFIVISMFLSVMGNVWLKHWSEVNSRYGSNPNAARYLAIYFALGIGSALATLIQTIVLWVFCTIHASKYLHNLMTNSVLRAPMTFFETTPIGRILNRFSNDIYKVDALLGRTFSQFFVNAVKVTFTITVICATTWQFIFIIIPLSVFYIYYQQYYLRTSRELRRLDSITRSPIYSHFQETLGGLATVRGYSQQKRFSHINQCRIDNNMSAFYPSINANRWLAYRLELIGSIIILGAATLSVFRLKQGTLTAGMVGLSLSYALQITQTLNWIVRMTVEVETNIVSVERIKEYADLKSEAPLIVEGHRPPKEWPSQGDIKFNNYSTRYRPELDLVLKHINIHIKPNEKVGIVGRTGAGKSSLTLALFRMIEASEGNIVIDNIAINEIGLYDLRHKLSIIPQDSQVFEGTVRENIDPINQYTDEAIWRALELSHLKEHVLSMSNDGLDAQLTEGGGNLSVGQRQLLCLARAMLVPSKILVLDEATAAVDVETDKVVQETIRTAFKDRTILTIAHRLNTIMDSDRIIVLDNGKVAEFDSPGQLLSDNKSLFYSLCMEAGLVNENGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLYAYMEKGITDLAPYPYDVPDYAGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRS

SEQ ID NO: 10 - protein, 1901 aa, Saccharomyces cerevisiae
MAGNLVSWACKLCRSPEGFGPISFYGDFTQCFIDGVILNLSAIFMITFGIRDLVNLCKKKHSGIKYRRNWIIVSRMALVLLEIAFVSLASLNISKEEAENFTIVSQYASTMLSLFVALALHWIEYDRSVVANTVLLFYWLFETFGNFAKLINILIRHTYEGIWYSGQTGFILTLFQVITCASILLLEALPKKPLMPHQHIHQTLTRRKPNPYDSANIFSRITFSWMSGLMKTGYEKYLVEADLYKLPRNFSSEELSQKLEKNWENELKQKSNPSLSWAICRTFGSKMLLAAFFKAIHDVLAFTQPQLLRILIKFVTDYNSERQDDHSSLQGFENNHPQKLPIVRGFLIAFAMFLVGFTQTSVLHQYFLNVFNTGMYIKSALTALIYQKSLVLSNEASGLSSTGDIVNLMSVDVQKLQDLTQWLNLIWSGPFQIIICLYSLYKLLGNSMWVGVIILVIMMPLNSFLMRIQKKLQKSQMKYKDERTRVISEILNNIKSLKLYAWEKPYREKLEEVRNNKELKNLTKLGCYMAVTSFQFNIVPFLVSCCTFAVFVYTEDRALTTDLVFPALTLFNLLSFPLMIIPMVLNSFIEASVSIGRLFTFFTNEELQPDSVQRLPKVKNIGDVAINIGDDATFLWQRKPEYKVALKNINFQAKKGNLTCIVGKVGSGKTALLSCMLGDRFRVKGFATVHGSVAYVSQVPWIMNGTVKENILFGHRYDAEFYEKTIKACALTIDLAILMDGDKTLVGEKGISLSGGQKARLSLARAVYARADTYLLDDPLAAVDEHVARHLIEHVLGPNGLLHTKTKVLATNKVSALSIADSIALLDNGEITQQGTYDEITKDADSPLWKLLNNYGKKNNGKSNEFGDSSESSVRESSIPVEGELEQLQKLNDLDFGNSDAISLRRASDATLGSIDFGDDENIAKREHREQGKVKWNIYLEYAKACNPKSVCVFILFIVISMFLSVMGNVWLKHWSEVNSRYGSNPNAARYLAIYFALGIGSALATLIQTIVLWVFCTIHASKYLHNLMTNSVLRAPMTFFETTPIGRILNRFSNDIYKVDALLGRTFSQFFVNAVKVTFTITVICATTWQFIFIIIPLSVFYIYYQQYYLRTSRELRRLDSITRSPIYSHFQETLGGLATVRGYSQQKRFSHINQCRIDNNMSAFYPSINANRWLAYRLELIGSIIILGAATLSVFRLKQGTLTAGMVGLSLSYALQITQTLNWIVRMTVEVETNIVSVERIKEYADLKSEAPLIVEGHRPPKEWPSQGDIKFNNYSTRYRPELDLVLKHINIHIKPNEKVGIVGRTGAGKSSLTLALFRMIEASEGNIVIDNIAINEIGLYDLRHKLSIIPQDSQVFEGTVRENIDPINQYTDEAIWRALELSHLKEHVLSMSNDGLDAQLTEGGGNLSVGQRQLLCLARAMLVPSKILVLDEATAAVDVETDKVVQETIRTAFKDRTILTIAHRLNTIMDSDRIIVLDNGKVAEFDSPGQLLSDNKSLFYSLCMEAGLVNENGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLYAYMEKGITDLAPYPYDVPDYAGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRSFCYENE

SEQ ID NO: 11 - protein, 1695 aa, Homo sapiens
MDLEGDRNGGAKKKNFFKLNNKSEKDKKEKKPTVSVFSMFRYSNWLDKLYMVVGTLAAIIHGAGLPLMMLVFGEMTDIFANAGNLEDLMSNITNRSDINDTGFFMNLEEDMTRYAYYYSGIGAGVLVAAYIQVSFWCLAAGRQIHKIRKQFFHAIMRQEIGWFDVHDVGELNTRLTDDVSKINEGIGDKIGMFFQSMATFFTGFIVGFTRGWKLTLVILAISPVLGLSAAVWAKILSSFTDKELLAYAKAGAVAEEVLAAIRTVIAFGGQKKELERYNKNLEEAKRIGIKKAITANISIGAAFLLIYASYALAFWYGTTLVLSGEYSIGQVLTVFSVLIGAFSVGQASPSIEAFANARGAAYEIFKIIDNKPSIDSYSKSGHKPDNIKGNLEFRNVHFSYPSRKEVKILKGLNLKVQSGQTVALVGNSGCGKSTTVQLMQRLYDPTEGMVSVDGQDIRTINVRFLREIIGVVSQEPVLFATTIAENIRYGRENVTMDEIEKAVKEANAYDFIMKLPHKFDTLVGERGAQLSGGQKQRIAIARALVRNPKILLLDEATSALDTESEAVVQVALDKARKGRTTIVIAHRLSTVRNADVIAGFDDGVIVEKGNHDELMKEKGIYFKLVTMQTAGNEVELENAADESKSEIDALEMSSNDSRSSLIRKRSTRRSVRGSQAQDRKLSTKEALDESIPPVSFWRIMKLNLTEWPYFVVGVFCAIINGGLQPAFAIIFSKIIGVFTRIDDPETKRQNSNLFSLLFLALGIISFITFFLQGFTFGKAGEILTKRLRYMVFRSMLRQDVSWFDDPKNTTGALTTRLANDAAQVKGAIGSRLAVITQNIANLGTGIIISFIYGWQLTLLLLAIVPIIAIAGVVEMKMLSGQALKDKKELEGAGKIATEAIENFRTVVSLTQEQKFEHMYAQSLQVPYRNSLRKAHIFGITFSFTQAMMYFSYAGCFRFGAYLVAHKLMSFEDVLLVFSAVVFGAMAVGQVSSFAPDYAKAKISAAHIIMIIEKTPLIDSYSTEGLMPNTLEGNVTFGEVVFNYPTRPDIPVLQGLSLEVKKGQTLALVGSSGCGKSTVVQLLERFYDPLAGKVLLDGKEIKRLNVQWLRAHLGIVSQEPILFDCSIAENIAYGDNSRVVSQEEIVRAAKEANIHAFIESLPNKYSTKVGDKGTQLSGGQKQRIAIARALVRQPHILLLDEATSALDTESEKVVQEALDKAREGRTCIVIAHRLSTIQNADLIVVFQNGRVKEHGTHQQLLAQKGIYFSMVSVQAGTKRQGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLYAYMEKGITDLAPYPYDVPDYAGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRSLLDALTLASSRGPLRKRSVAVAKAKPEFSISPDSLS

SEQ ID NO: 12 - protein, 1941 aa, Rattus rattus
MSLSFCGNNISSYNIYHGVLQNPCFVDALNLVPHVFLLFITFPILFIGWGSQSSKVQIHHNTWLHFPGHNLRWILTFALLFVHVCEIAEGIVSDSQRASRHLHLFMPAVMGFVATTTSIVYYHNIETSNFPKLLLALFLYWVMAFITKTIKLVKYWQLGWGMSDLRFCITGVMVILNGLLMAVEINVIRVRRYVFFMNPQKVKPPEDLQDLGVRFLQPFVNLLSKATYWWMNTLIISAHRKPIDLKAIGKLPIAMRAVTNYVCLKEAYEEQKKKAADHPNRTPSIWLAMYRAFGRPILLSSTFRYLADLLGFAGPLCISGIVQRVNEPKNNTTRFSETLSSKEFLENAHVLAVLLFLALILQRTFLQASYYVTIETGINLRGALLAMIYNKILRLSTSNLSMGEMTLGQINNLVAIETNQLMWFLFLCPNLWAMPVQIIMGVILLYNLLGSSALVGAAVIVLLAPIQYFIATKLAEAQKSTLDYSTERLKKTNEILKGIKLLKLYAWEHIFCKSVEETRMKELSSLKTFALYTSLSIFMNAAIPIAAVLATFVTHAYASGNNLKPAEAFASLSLFHILVTPLFLLSTVVRFAVKAIISVQKLNEFLLSDEIGEDSWRTGEGTLPFESCKKHTGVQSKPINRKQPGRYHLDNYEQARRLRPAETEDVAIKVTNGYFSWGSGLATLSNIDIRIPTGQLTMIVGQVGCGKSSLLLAILGEMQTLEGKVYWNNVNESEPSFEATRSRSRYSVAYAAQKPWLLNATVEENITFGSSFNRQRYKAVTDACSLQPDIDLLPFGDQTEIGERGINLSGGQRQRICVARALYQNTNIVFLDDPFSALDIHLSDHLMQEGILKFLQDDKRTVVLVTHKLQYLTHADWIIAMKDGSVLREGTLKDIQTKDVELYEHWKTLMNRQDQELEKDMEADQTTLERKTLRRAMYSREAKAQMEDEDEEEEEEEDEDDNMSTVMRLRTKMPWKTCWWYLTSGGFFLLFLMIFSKLLKHSVIVAIDYWLATWTSEYSINDPGKADQTFYVAGFSILCGAGIFLCLVTSLTVEWMGLTAAKNLHHNLLNKIILGPIRFFDTTPLGLILNRFSADTNIIDQHIPPTLESLTRSTLLCLSAIGMISYATPVFLIALAPLGVAFYFIQKYFRVASKDLQELDDSTQLPLLCHFSETAEGLTTIRAFRHETRFKQRMLELTDTNNIAYLFLSAANRWLEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDATVTEGGENFSVGQRQLFCLARAFVRKSSILIMDEATASIDMATENILQKVVMTAFADRTVVTIAHRVSSIMDAGLVLVFSEGILVECDTGPNLLQHKNGLFSTLVMTNKGGGGGGMLSRKGIIPEEYVLTRLAEDPAEPRYRTRERRARFVSKKGNCNVAHKNIREQGRFLQDVFTTLVDLKWPHTLLIFTMSFLCSWLLFAMVWWLIAFAHGDLAPGEGTNVPCVTSIHSFSSAFLFSIEVQVTIGFGGRMVTEECPLAILILIVQNIVGLMINAIMLGCIFMKTAQAHRRAETLIFSKHAVITLRHGRLCFMLRVGDLRKSMIISATIHMQVVRKTTSPEGEVVPLHQVDIPMENGVGGNGIFLVAPLIIYHVIDSNSPLYDLAPSDLHHHQDLEIIVILEGVVETTGITTQARTSYLADEILWGQRFVPIVAEEDGRYSVDYSKFGNTIKVPTPLCTARQLDEDRSLLDALTLASSRGPLRKRSVAVAKAKPKFSISPDSLS

SEQ ID NO: 13 - DNA, 25 bp, artificial sequence
  MRP1 forward primer
ggtcacccatgagcttctttgagcg

SEQ ID NO: 14 - DNA, 33 bp, artificial sequence
  MRP1 fusion reverse prrimer
gcctccacctccacctcccaccaagccggcgtc

SEQ ID NO: 15 - DNA, 30 bp, artificial sequence
  Kir6.2 reverse primer
caagtgagtgcttaagtgaggaactgcaac

SEQ ID NO: 16 - DNA, 36 bp, artificial sequence
  Kir6.2 fusion forward primer
ggaggtggaggtggaggcatgctgtcccgaaagggc

SEQ ID NO: 17 - DNA, 20 bp, artificial sequence
  SUR1 forward primer
ccgtgccttcaggtacgagg

SEQ ID NO: 18 - DNA, 38 bp, artificial sequence
  SUR1 fusion reverse primer
gcctccacctccacctcccttgtccgcacggacaaagg

SEQ ID NO: 19 - DNA, 20 bp, artificial sequence
  SUR2A forward primer
ttcaagcaacgcatgctgga

SEQ ID NO: 20 - DNA, 36 bp, artificial sequence
  SUR2A fusion reverse primer
gcctccacctccacctcccttgttggtcatcaccaa

SEQ ID NO: 21 - DNA, 20 bp, artificial sequence
  YCF1 forward primer
ccctctatcaatgctaaccg

SEQ ID NO: 22 - DNA, 36 bp, artificial sequence
  YCF1 fusion reverse primer
gcctccacctccacctccattttcattgaccaaacc

SEQ ID NO: 23 - protein, 9 aa, artificial sequence
  HA epitope
YPYDVPDYA

SEQ ID NO: 24 - protein, 11 aa, artificial sequence
  -
DLYAYMEKGIT

SEQ ID NO: 25 - protein, 6 aa, artificial sequence
  export signal
FCYENE